(19) United States
(12) Patent Application Publication
(10) Pub. No.: US 2010/0040672 A1
(43) Pub. Date: Feb. 18, 2010

(54) DELIVERY OF THERAPEUTICS

(75) Inventors: Dean Ho, Chicago, IL (US); Robert Lam, Evanston, IL (US); Mark Chen, Chicago, IL (US); Houjin Huang, Evanston, IL (US); Erik Pierstorff, Highland Park, IL (US); Erik Robinson, Chicago, IL (US)

Correspondence Address: Casimir Jones, S.C., 2275 DEMING WAY, SUITE 310, MIDDLETON, WI, 53562, US

(73) Assignee: Northwestern University, Evanston, IL (US)

(21) Appl. No.: 12/481,400

(22) Filed: Jun. 9, 2009

Related U.S. Application Data
(60) Provisional application No. 61/059,979, filed on Jun. 9, 2008.
(60) Provisional application No. 61/059,976, filed on Jun. 9, 2008.

(51) Int. Cl. A61K 9/70; A61K 31/56; A61K 31/351

(52) U.S. Cl. 424/443; 514/179; 514/459

(57) ABSTRACT

The present invention provides materials and devices for the controlled release of therapeutics, and methods for uses thereof.

The present application claims priority to U.S. Provisional Patent Application Ser. No. 61/059,976 filed Jun. 9, 2008, and U.S. Provisional Patent Application Ser. No. 61/059,979 filed Jun. 9, 2008, which are herein incorporated by reference in their entireties.

### STATEMENT REGARDING FEDERALLY SPONSORED RESEARCH OR DEVELOPMENT

This invention was made with government support under grant no. U54 AI065359-02 awarded by the National Institutes of Health, and grant no. DMI-0327077 awarded by the National Science Foundation. The government has certain rights in the invention.

### FIELD OF THE INVENTION

The present invention provides materials and devices for the controlled release of therapeutics, and methods for uses thereof.

### BACKGROUND

Numerous synthetic and natural nanoscale carriers have been developed and investigated for modulating therapeutic release. These include, but are not limited to polymer-protein conjugates, liposomes, micelles, dendrimers, polyelectrolyte films, copolypeptides, carbon nanotubes and variants of the above. The family of poly-p-xylenes, commonly known as Parylene, is an accepted coating for medically implanted devices due to its biocompatibility (Eskin et al. Journal of Biomedical Materials Research, 1976, 10, 113., Fontaine et al. 1996, 3, 276., herein incorporated by reference in their entireties) and ability to form a conformal barrier (Fortin & Lu. Chemical Vapor Deposition Polymerization The Growth and Properties of Parylene Thin Films; Kluwear: Norwell, 2004., herein incorporated by reference in its entirety) between the medical device and exterior environment. Efforts have been made to apply alternative drug-conjugated polymeric coatings to the Parylene surface (Westedt et al. Journal of Controlled Release 2006, 111, 235., Unger et al. Journal of Controlled Release 2007, 117, 312., herein incorporated by reference in their entireties). While these developments have proved useful, improved compositions and methods are needed for delivery of therapeutic materials.

### SUMMARY

The present invention provides materials and devices for the controlled release of therapeutics, and methods for uses thereof. In some embodiments, the present invention provides nanofilms, functionalized nanodiamonds, nanodiamond clusters, bilayer carrier/delivery elements, hydrogel delivery/carrier elements, and/or combinations thereof for the controlled release for the controlled release of therapeutics.

The present invention provides several classes of therapeutic delivery systems, devices, methods, materials, and compositions: (1) a nanofilm comprising: a base layer, wherein the base layer is composed of Parylene A, an elution layer, wherein, the elution layer is composed of Parylene A, and a therapeutic layer, wherein the therapeutic layer is composed of at least one therapeutic agent, and wherein the therapeutic layer is between the base layer and the elution layer; (2) a nanofilm comprising: a nanodiamond layer, wherein the nanodiamond layer is comprised of nanodiamonds functionalized with at least one therapeutic agent, a base layer, and an elution layer, wherein, the nanodiamond layer is between the base layer and the elution layer; (3) a composition comprising: a nanodiamond element, wherein the nanodiamond element comprises nanodiamonds functionalized with at least one therapeutic agent, and a carrier element, wherein the nanodiamond element is contained within the carrier element; (4) devices, methods, materials, and compositions comprising combinations, alterations, and/or modifications of all or portions (1)-(3) with elements disclosed herein or known to those of skill in the art.

In some embodiments, the present invention relates to localized nanodiamond elution through a nanofilm device. In some embodiments, the present invention provides nanodiamond-embedded nanofilm devices and methods for therapeutic uses thereof. In some embodiments, nanodiamonds functionalized with at least one therapeutic agent are embedded between two or more polymer layers, such as a base layer and a semi-permeable layer (e.g. elution layer). In some embodiments the base layer is thick (e.g. thicker than the semipermeable layer), rough, and impermeable. In some embodiments, the semi-permeable layer is thin (e.g. ultra-thin, nanometer scale, etc.). In some embodiments, the semi-permeable layer comprises nanopores through which the functionalized nanodiamonds are capable of eluting. In some embodiments, a nanofilm device can be used to deliver therapeutics to a subject through the elution of the therapeutic-functionalized nanodiamonds from the nanofilm (e.g. onto a surface of the subject (e.g. skin, mucous membrane, etc.) into a subject (e.g. body cavity, blood, etc.)).

Nanodiamonds (NDs) possess several characteristics that make them suitable for advanced drug delivery. Due to their high surface area to volume ratio and non-invasive dimensions, extremely high loading capacities of therapeutic are achievable. In addition, NDs are capable of interfacing with virtually any therapeutic molecule via physical interactions due to tailorable surface properties and compositions.

Embodiments of the present invention provide a nanofilm composition comprised of a nanodiamond layer, a base layer, and a semi-permeable layer. In some embodiments, the nanodiamond layer lies between the base layer and the semi-permeable layer, and is comprised of nanodiamonds functionalized with at least one therapeutic agent.

In some embodiments of the present invention, a therapeutic agent functionalized with nanodiamonds comprises, but is not limited to: sirtuin activators, cytokines (e.g. interferons of all kinds, e.g. alpha, beta, gamma, etc.), thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents. In some embodiments where the therapeutic agent is an anti-inflammatory compound, the therapeutic agent can be dexamethasone, glucocorticoid, or an LXR agonist. In some embodiments where the therapeutic agent is an anticancer chemotherapeutic agent, the therapeutic agent can be doxorubicin (DOX).

In some embodiments of the present invention, the base layer may comprise Parylene (e.g. Parylene A or Parylene C), and more particularly may comprise Parylene C. Likewise, the semi-permeable layer may comprise Parylene (e.g. Parylene A or Parylene C), and more particularly may comprise Parylene C. In embodiments in which the base and/or semi-permeable layers are comprised of Parylene, the Parylene may be treated with oxygen plasma. In some embodiments, the base and/or semi-permeable layers may contain added CO₃⁻ and carbonyl (C═O) groups. Other treatments understood by one in the art may also be made to the Parylene material.

Parylene refers to a variety of polyxylene polymers marketed by several providers, including Para Tech Coating, Inc., Specialty Coating Systems, Inc., and others. Parylene N is a polymer manufactured from di-p-xylylene, a dimer synthesized from p-xylene. Di-p-xylylene, more properly known as (2.2)paracyclophane, is made from p-xylene in several steps involving bromination, amination and elimination. There are a number of derivatives and isomers of Parylene, but only a few are typically used commercially, e.g. Parylene C and Parylene D.

In some embodiments, a single layer of the nanofilm may be designed to have a thickness from about 1 nm to about 10 nm, desirably less than about 4 nm, although dimensions outside this range are contemplated. However, the nanofilm may include multiple layers (e.g., from about 2 to about 10 layers) of the therapeutic agent complexes, wherein each layer has a thickness from about 1 to about 10 nm (e.g., about 4 nm or less). The functionalized nanodiamonds may be approximately 2-8 nm in diameter, although other dimensions are contemplated.

In some embodiments of the present invention, the semi-permeable layer contains nanopores. The nanodiamond layer is configured to elute through the nanopores in the semi-permeable layer. In some embodiments, nanodiamonds functionalized with at least one therapeutic agent are configured to elute through nanopores in the semi-permeable layer, but the nanodiamond layer is incapable of elution through the base layer.

In some embodiments of the present invention, the nanofilm composition, comprised of a nanodiamond layer, a base layer and a semi-permeable layer, is flexible. In some embodiments, the nanofilm composition is fashioned as a transversal patch.

In some embodiments, the present invention provides a medical device with one or more of its surfaces coated with any of the nanofilm compositions described herein. The medical device may be implantable. In some embodiments the medical device contains an electrode. The nanofilm coatings of the present invention may be used on a variety of medical substrates, including an implantable medical device. Such medical devices may be made of a variety of biocompatible materials including, but not limited to, polymers and metals. Medical substrates onto which the nanofilms may be coated include, neural/cardiovascular/retinal implants, leads and stents, and dental implants (e.g., nanofilms to seed bone growth). In some embodiments, the nanofilm may be coated onto the electrode of an implantable medical device. In fact, coating the present nanofilms onto an electrode is contemplated to provide important medical advantage because the nanofilm is contemplated to prevent or minimize bio-fouling which often begins at the site of a metal electrode. In addition, unlike more conventional implant coatings, the present nanofilms may be made thin enough that they do not interfere with electrode function (e.g., electrical conductivity or redox reactions at electrodes). Other medical device uses and configurations will be understood by one skilled in the art using the principles described herein.

In some embodiments, the present invention provides a method of delivering a therapeutic agent to a target site in a subject, in which any nanofilm composition described herein, is administered to the subject near a target site. Elution of the therapeutic agent from the nanofilm device delivers the therapeutic agent to the target site. The nanofilm composition may comprise a transdermal patch or coat a medical device, or other desired application. In embodiments where the nanofilm coats a medical device, that medical device may be implantable within a subject. The therapeutic agent delivered by the method of the present invention includes, but is not limited to: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents.

A semi-permeable layer of a device of the present invention may be selected to optimize drug elution rates, so as to provide optimal drug delivery for a particular drug type and therapy type. Selection of the size and shape of the nanopores and the nature of the polymer material may be tailored to optimize drug release characteristics.

In some embodiments of the present invention, the devices are used to harness intelligent drug release activity by releasing algorithmically/search scheme derived optimum concentrations of drugs for any medical or cosmetic to deliver a personalized medical treatment strategy. In some embodiments, this device is used to harness any search algorithm, including but not limited to simulated annealing, genetic algorithms, ant colony optimization, and the Gur Game including all other algorithms. In some embodiments the devices are functionalized with any therapeutically relevant molecule as well as sequestering matrix to enable slow and targeted release based upon a broad range of stimuli including but not limited to temperature, pH, light, salt concentrations, chemical stimuli, etc. In some embodiments therapeutics that are released include but are not limited to conventional chemically synthesized drugs for anti-inflammation, chemotherapy, anti-angiogenesis, wound/burn healing, pain management, membrane repair, anti-coagulation, anti-infection/anti-bacterial/anti-viral applications, etc. In some embodiments a device of the present invention carries RNAi-based therapeutics and stabilizes RNAi molecules to enable sustained/long-term release with enhanced efficacy, as well as protein, small molecule, and antibody-based therapies, etc. In some embodiments the present invention also has applicability towards cosmetic applications by delivering anti-wrinkle, anti-acne, acid treatment, collagen, micro/nanobead, moisturizing, traditional eastern medicine ingredients as well as virtually any other cosmetic agent that can be employed. In some embodiments sequestering matrices that can be carried include nanodiamonds, block copolymers, polymer matrices, crosslinked networks, hydrogels, polymer amphiphiles, peptide amphiphiles, nanotubes made of carbon or polymers, carbon nanohorns, as well as the entire spectrum of carbon-based nanomaterials, metallic nanoparticles, silica nanoparticles, protein-based nanoparticles, nucleic acid-based nanoparticles, etc.

In some embodiments, the present invention relates to delivery of therapeutics through a functionalized nanofilm device. In some embodiments, the present invention provides an amine functionalized poly-p-xylene (Parylene) nanofilm device and methods for localized delivery of therapeutics thereof. In some embodiments, a layer comprised of at least one therapeutic agent is embedded between two or more Parylene A layers, such as a base layer and an elution layer. In some embodiments, the base layer is thick (e.g. thicker than an elution layer) and/or impermeable. In some embodiments, the elution layer (e.g. semi-permeable layer, permeable layer, etc.) is thin and/or contains openings (e.g., pores, pinholes, etc.) through which the therapeutic agent or agents are capable of eluting. In some embodiments, a Parylene A nanofilm device can be used to deliver therapeutics to a subject through the elution of the therapeutic agent from the nanofilm. In some embodiments, the amine functionalized Parylene A provides reactive groups on the surface of a Parylene A nanofilm. It is contemplated that the availability of free amine groups on the Parylene A surface provides a range of modifications which can be incorporated into the nanofilm, for example, through the conjugation of other molecules to the amine groups.

In some embodiments, the present invention provides a nanofilm composition comprised of a Parylene A base layer, a Parylene A elution layer, and a therapeutic layer. The therapeutic layer lies between the base layer and the elution layer, and is comprised of at least one therapeutic agent. In some embodiments of the present invention, the therapeutic agent within the therapeutic layer comprises, but is not limited to: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents. In some embodiments where the therapeutic agent is an anti-inflammatory compound, the therapeutic agent can be dexamethasone, glucocorticoid, or an LXR agonist. In some embodiments where the therapeutic agent is an anticancer chemotherapeutic agent, the therapeutic agent can be doxorubicin (DOX).

In some embodiments, a single layer of the nanofilm may be designed to have a thickness from about 1 nm to about 10 nm, desirably less than about 4 nm, although dimensions outside this range are contemplated (e.g. 15 nm, 25 nm, 50 nm, 100 nm, etc.). However, the nanofilm may include multiple layers (e.g., from about 2 to about 10 layers, 5 to 15 layers, 10 to 50 layers, etc.) of the therapeutic agent complexes, wherein each layer has a thickness from about 1 to about 10 nm (e.g., about 4 nm or less), although dimensions outside this range are contemplated.

In some embodiments of the present invention, the elution layer contains openings (e.g., pinholes, pores, etc.). In embodiments where the elution layer contains pinholes, the elution layer may exhibit some degree of permeability (e.g. semi-permeable, permeable, etc.). The therapeutic layer can be provided with a therapeutic agent or agents that elute through pinholes in the elution layer. In some embodiments, at least one therapeutic agent is configured to elute through pinholes in the elution layer. In some embodiments, the therapeutic layer, and any therapeutic agent or agents therein, are incapable of elution through the base layer. An elution layer of a device of the present invention may be selected to optimize drug elution rates, so as to provide optimal drug delivery for a particular drug type and therapy type. Selection of the size and shape of the pinholes and the nature of the polymer material may be tailored to optimize drug release characteristics.

In some embodiments, the present invention provides a nanofilm composition comprising: (a) a base layer, wherein the base layer is composed of Parylene A, (b) an elution layer, wherein, the elution layer is composed of Parylene A, and (c) a therapeutic layer, wherein the therapeutic layer is composed of at least one therapeutic agent, and wherein the therapeutic layer is between the base layer and the elution layer. In some embodiments, at least one therapeutic agent is selected from the group consisting of: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents. In some embodiments, the anti-inflammatory compound is dexamethasone (DEX), glucocorticoid, or an LXR agonist. In some embodiments, the anticancer chemotherapeutic agent is doxorubicin (DOX). In some embodiments, the elution layer is semi-permeable. In some embodiments, the therapeutic layer is configured to elute through said elution layer.

In some embodiments, the present invention provides a nanofilm composition comprising: (a) a nanodiamond layer, wherein the nanodiamond layer is comprised of nanodiamonds functionalized with at least one therapeutic agent, (b) a base layer, and (c) an elution layer, wherein, the nanodiamond layer is between the base layer and the elution layer. In some embodiments, the said at least one therapeutic agent is selected from the group consisting of: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents. In some embodiments, the anti-inflammatory compound is dexamethasone, glucocorticoid, or an LXR agonist. In some embodiments, the anticancer chemotherapeutic agent is doxorubicin (DOX). In some embodiments, the base layer comprises a Parylene compound. In some embodiments, the elution layer comprises a Parylene compound. In some embodiments, the nanodiamond layer is configured to elute through the elution layer.

In some embodiments, the present invention provides a composition comprising: (a) a nanodiamond element, wherein the nanodiamond element is comprised of nanodiamonds functionalized with at least one therapeutic agent; and (b) a carrier element, wherein the nanodiamond element is contained within the carrier element. In some embodiments, the at least one therapeutic agent is selected from the group consisting of: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents. In some embodiments, the anti-inflammatory compound is dexamethasone, glucocorticoid, or an LXR agonist. In some embodiments, the anticancer chemotherapeutic agent is doxorubicin (DOX). In some embodiments, the carrier element comprises a PEG hydrogel. In some embodiments, the carrier element is semi-permeable. In some embodiments, the therapeutic element is configured to elute through the carrier element.

### DESCRIPTION OF FIGURES

The foregoing summary and detailed description may be better understood when read in conjunction with the accompanying drawings which are included by way of example and not by way of limitation.

FIG. 1 shows a schematic illustrating the chemical structure Parylene A, and a therapeutic (drug) eluting Parylene A nanofilm device.

FIG. 2 shows graphs of gene expression of a) inflammatory cytokine Interleukin-6 (IL-6) and b) inflammatory cytokine Tumor Necrosis Factor-μ (TNF-α). LPS stimulation of RAW 2647 macrophages occurred during the last 4 hours of a 24 hour incubation. 1)−LPS; cellular growth on a glass slide, 2)−LPS; comparable growth on Parylene A coated slide, 3)+LPS; cellular response on Parylene A substrate, 4)+LPS; resultant action of a DEX film upon Parylene A coating, 5)+LPS; Parylene A bi-layer, and 6)+LPS; Parylene A bi-layer incorporated with a DEX film. Arrows indicate DEX mediated decrease.

FIG. 3 shows an electrophoretic gel depicting doxorubicin induced DNA fragmentation of RAW 264.7 macrophages. 1) Cells grown upon plain glass slide. 2) Addition of aqueous doxorubicin to the previously indicated conditions showing fragmentation of DNA indicative of cells actively undergoing apoptosis. 3) Parylene A substrate showing no apoptotic response to the amine functionalized surface. 4) DOX film applied to previous sample revealing the ladder banding pattern of DNA fragmentation. 5) Parylene A bi-layer revealing no significant apoptotic response to the additional secondary eluting layer. 6) DOX loaded into the Parylene A bi-layer showing apoptotic fragmentation of DNA.

FIG. 4 shows (a) additive spectroscopic scans showing the complete release of DOX from control samples consisting of DOX applied to a base layer of Parylene A deposited on glass disks, (b) additive spectroscopic scans showing the gradual release of DOX from a pinhole sample consisting of DOX introduced between alternating amounts Parylene A deposited on glass disks, and (c) comparison of peak absorbance values (480 nm) of DOX elution in PBS over 4 hours.

FIG. 5 shows a photo of a Parylene A film revealing its micron thin profile and flexibility.

FIG. 6 shows a) an illustrated schematic of a DOX-ND encapsulated Parylene C nanofilm. b) A photograph of DOX-ND encapsulated Parylene C nanofilms with a 10 g base layer, or varied size and shape. c) A demonstration of the flexibility of DOX-ND encapsulated Parylene C nanofilm.

FIG. 7 shows atomic force microscopy (AFM) images of Parylene C: a) native Parylene C (roughness of 6.245 nm); b) plasma-treated Parylene C (roughness of 9.291 nm); c) DOX-NDs deposited on a plasma treated layer of Parylene C; d) DOX-NDs deposited on a plasma treated layer of Parylene C, and covered with an additional thin layer of Parylene C.

FIG. 8 shows DOX-ND release assessment data. a) Sample UV-vis spectra of eluate collected at 24 hour intervals for samples uncovered with top eluting Parylene element. b) 8 day trials performed for Parylene covered and uncovered samples (optical absorbance measured at 480 nm. c) a long term trial where eluate was collected at various time points and optical absorbance is measured at 480 nm. d) Spectroscopy data at 480 nm plotted against respective DOX concentration.

FIG. 9 shows gel electrophoresis assay of DNA from RAW 264.7 murine macrophages incubated for 16 hours (lanes 1-4) and 20 hours (lanes 5-8) on glass (lands 1, 5), DOX-ND on Parylene C (lanes 2, 6), DOX-ND sandwiched between a base layer and pinhole layer of Parylene C (lanes 3, 7), and Parylene with 2.5 μg DOX. Degrees of banding correlate to different stage of apoptosis induced by DOX incubation.

FIG. 10 shows ND-DOX elution from Parylene thin films over 28 days measured via peak absorbance.

FIG. 11 shows triplicate trials of ND deficient (A) and ND embedded (B) 50% PEGDA hydrogels. Hydrogels contained 250 μg/mL of DOX in solution prior to submersion.

FIG. 12 shows sample hydrogels: rows 1, 2: DOX-PEGDA hydrogels before and after 24 hour incubation in pure water, rows 3, 4: DOX-ND:PEGDA hydrogels before and after 10 day incubation, column A: 50% PEGDA with 250 μg/mL of DOX, column B: 25% PEGDA with 250 μg/mL DOX, column C: 50% PEGDA with 125 μg/mL DOX, column D: 25% PEGDA with 125 μg/mL DOX.

FIG. 13 shows UV-vis spectra of eluate collected every 24 hours from hydrogels of indicated drug and ND composition in nanopure water or PBS.

FIG. 14 shows ESEM images of (a) PEGDA, (b) DOX:PEGDA and DOX-ND:PEGDA hydrogels at 1000× magnification and 1.20 Torr; scale bars=50 μm.

### DETAILED DESCRIPTION

The present invention provides compositions, materials, and devices for the controlled release of therapeutics, and methods for uses thereof. In particular, the present invention provides a therapeutic element and a carrier and/or delivery element. In some embodiments, a carrier/delivery element provides a means for applying one or more therapeutic elements to a device, surface, material, composition, tissue, or subject. In some embodiments, a carrier/delivery element provides the controlled release of one or more therapeutic elements onto, into, or from the surface of a device, surface, material, composition, tissue, or subject upon which it is applied.

### A. Carrier/Delivery Element

In some embodiments, the present invention provides a carrier/delivery element. In some embodiments, a carrier/delivery element provides a barrier, surface, or material to contain, encapsulate, sequester, or confine a therapeutic element. In some embodiments, a carrier/delivery element is configured to allow the controlled release of a therapeutic element. In some embodiments, a carrier/delivery element comprises a layer above and/or adjacent to a therapeutic layer. In some embodiments, a carrier/delivery element comprises a bilayer above and below to a therapeutic layer. In some embodiments, a carrier/delivery element comprises a bilayer which surrounds a therapeutic layer. In some embodiments, a therapeutic layer resides between the individual layers of a bilayer of a carrier/delivery element. In some embodiments, a bilayer comprises a base layer and an elution or semi-permeable layer. In some embodiments, a carrier/delivery element comprises a matrix or material within which a therapeutic element is contained. In some embodiments, a carrier/delivery element is porous, permeable, and/or semi-permeable. In some embodiments, a carrier/delivery element is configured to provide controlled release of a therapeutic element from the carrier/delivery element. In some embodiments, a carrier/delivery element comprises one or more matrix elements and one or more bilayer elements. In some embodiments, a matrix element resides between the individual layers of a bilayer. In some embodiments, a matrix element embedded with a therapeutic element resides between a base layer and elution layer of a bilayer element.

In some embodiments, a carrier/delivery element comprises a film, thin-layer film, or nanofilm. In some embodiments, a carrier/delivery element or elements may be of any desired thickness (e.g. 0.1 nm . . . 0.2 nm . . . 0.5 nm . . . 1.0 nm . . . 2.0 nm . . . 5.0 nm . . . 10 nm . . . 20 nm . . . 50 nm . . . 100 nm . . . 200 nm . . . 500 nm . . . 1 mm . . . 2 mm . . . 5 mm . . . 1 cm, and thicknesses therein, etc.). In some embodiments, a carrier/delivery element can be configured in any shape, dimensions, etc.

### Bilayer Carrier/Delivery Element

In some embodiments, the present invention provides a bilayer carrier/delivery element. In some embodiments, a carrier/delivery element comprises a base layer and a top layer (e.g. elution layer, semi-permeable layer, release layer, degradable layer, etc.). In some embodiments, the present invention provides a therapeutic layer. In some embodiments, a therapeutic layer resides between a base layer and a top layer. In some embodiments, one or both of a base layer and a top layer are porous, permeable, and/or semi-permeable (e.g. permeable to one or more therapeutics of a therapeutic layer). In some embodiments, one or both of a base layer and a top layer are impermeable (e.g. impermeable to one or more therapeutics of a therapeutic layer). In some embodiments, the present invention provides an impermeable base layer and a permeable or semi-permeable elution layer. In some embodiments, a therapeutic element (e.g. therapeutic layer) resides between an impermeable base layer and a top elution layer (e.g. permeable layer or semi-permeable layer). In some embodiments, a top elution layer is configured to allow the controlled elution of a therapeutic. In some embodiments, a top elution layer is configured to allow the controlled elution of a therapeutic from the therapeutic layer through the elution layer. In some embodiments, a base layer is configured to allow elution. In some embodiments, a base layer is configured to resist elution.

In some embodiments, one or more layers (e.g. top layer, base layer, elution layer, etc.) of the present invention comprise one or more polymers including, but not limited to polyacrylates, polyamides, polyesters, polycarbonates, polyimides, polystyrenes, acrylonitrile butadiene styrene (ABS), polyacrylonitrile (PAN) or Acrylic, polybutadiene, poly(butylene terephthalate) (PBT), poly(ether sulfone) (PES, PES/PEES), poly(ether ether ketone)s (PEEK, PES/PEEK), polyethylene (PE), poly(ethylene glycol) (PEG), poly(ethylene terephthalate) (PET), polypropylene (PP), polytetrafluoroethylene (PTFE), styrene-acrylonitrile resin (SAN), poly(trimethylene terephthalate) (PTT), polyurethane (PU), polyvinyl butyral (PVB), polyvinylchloride (PVC), polyvinylidenedifluoride (PVDF), poly(vinyl pyrrolidone) (PVP), poly-p-xylenes, derivatives thereof, substituents thereof, combinations thereof, similar polymers, etc.

In some embodiments, one or more layers (e.g. top layer, base layer, elution layer, etc.) of the present invention comprise a nanofilm. In some embodiments, the thickness of a nanofilm is less than about 100 nanometers (e.g. <100 nm, <50 nm, <20 nm, <10 nm, <5.0 nm, <2.0 nm, <1.0 nm, <0.5 nm, <0.2 nm, <0.1 nm, etc.). In some embodiments, the thickness of a nanofilm is greater than about 0.1 nanometers (e.g. >0.1 nm, >0.2 nm, >0.5 nm, >1.0 nm, >2.0 nm, >5.0 nm, >10 nm, >20 nm, >50 nm, etc.). In some embodiments, a nanofilm may be permeable, semi-permeable, or impermeable. In some embodiments, a nanofilm has a filtration function, allowing certain species to pass through the nanofilm. In some embodiments, a nanofilm has a controlled filtration function, allowing species to pass through the nanofilm at a defined rate (e.g. based on pore size, pore frequency, etc.). In some embodiments, a nanofilm is permeable only to particular species in particular fluid and/or species smaller than the particular species. In some embodiments, a nanofilm has a molecular weight cut-off. In some embodiments, a nanofilm has a high permeability for certain species in a certain solvent. In some embodiments, a nanofilm has a low permeability for certain species in a certain solvent. In some embodiments, a nanofilm has a high permeability for certain species and low permeability for other species in a certain solvent. In some embodiments, a nanofilm composition, material, and/or device is a made up of two or more layers of nanofilm. In some embodiments, a spacing layer may be used between any two nanofilm layers. In some embodiments, spacing layers may include a polymer layer, gel layer, hydrogel layer, therapeutic layer, void layer, etc. In some embodiments, a nanofilm, or other material of the present invention, is deposited on a substrate (e.g. surface, material, composition, device, etc.), which may be porous or non-porous. In some embodiments, a nanofilm, or other material of the present invention, has surface attachment groups, and may be covalently bonded to a substrate through surface attachment groups, or bonded to a substrate through ionic interactions. In some embodiments, surface attachment groups provide contact or links between multiple layers of nanofilm, or between nanofilm layers and other layers (e.g. polymer layer, gel layer, hydrogel layer, therapeutic layer, etc.).

In some embodiments, a layer or layers of the present invention provide nanopores which allow controlled elution of a therapeutic. In some embodiments, nanopores of the present invention are greater than 0.1 nm in diameter (e.g. >0.1 nm, >0.2 nm, >0.5 nm, >1.0 nm, >2.0 nm, >5.0 nm, >10 nm, >20 nm, >50 nm, >100 nm, >200 nm, >500 nm, >1 mm, etc.). In some embodiments, nanopores of the present invention are less than 2 mm in diameter (e.g. <2 mm, <1 mm, <500 nm, <200 nm, <100 nm, <50 nm, <20 nm, <10 nm, <5.0 nm, <2.0 nm, <1.0 nm, <0.5 nm, <0.2 nm, <0.1 nm, etc.). In some embodiments, nanopores of the present invention have diameters between 0.1 nm and 1 mm (e.g. 0.1 nm, 0.2 nm, 0.3 nm, 0.4 nm, 0.5 nm, 0.6 nm, 0.7 nm, 0.8 nm, 0.9 nm, 1.0 nm, 1.1 nm, 1.2 nm, 1.3 nm, 1.4 nm, 1.5 nm, 1.6 nm, 1.7 nm, 1.8 nm, 1.9 nm, 2.0 nm, 3.0 nm, 4.0 nm, 5.0 nm, 6.0 nm, 7.0 nm, 8.0 nm, 9.0 nm, 10 nm, 20 nm, 30 nm, 40 nm, 50, nm, 100, nm, 200, nm, 500, nm, 1 mm, and diameters therein). In some embodiments, an elution layer, top layer, base layer, permeable layer, porous layer, and/or semi-permeable layer of the present invention comprises nanopores.

In some embodiments, a carrier/delivery element is a nanofilm composition comprising a base layer, therapeutic layer, and an elution layer, wherein the therapeutic layer resides between the base layer and the elution layer. In some embodiments, the base layer is permeable, semi-permeable, or impermeable. In some embodiments, the base layer is comprised of any materials disclosed herein or any other suitable materials. In some embodiments, the base layer is configured for interaction with a substrate (e.g. surface, material, composition, device, etc.). In some embodiments, a base layer provides functional groups or other characteristics (e.g. adhesive) known to those in the art for interaction with a substrate. In some embodiments, the base layer provides stable interaction with a substrate. In some embodiments, the base layer interacts with a substrate to modify the surface of the substrate. In some embodiments, the present invention provides a permeable or semi-permeable elution layer. In some embodiments, the elution layer is comprised of any materials disclosed herein or any other suitable materials. In some embodiments, the elution layer is permeable or semi-permeable to one or more molecules, macromolecules (e.g. peptides, lipids, nucleic acids, etc.), compositions, therapeutics, drugs, small molecules, etc. contained within an underlying layer (e.g. therapeutic layer). In some embodiments, the elution layer provides release (e.g. controlled release) of one or more contents of the underlying layer (e.g. therapeutic layer). In some embodiments, the elution layer provides release (e.g. controlled release) of one or more contents of the underlying layer (e.g. therapeutic layer) through pores in the elution layer. In some embodiments, degradation of the elution layer by environmental factors (e.g. dissolving into solvent, hydrolysis, etc.) provides release (e.g. controlled release of one or more contents of the underlying layer (e.g. therapeutic layer). In some embodiments, the base layer provides positioning of the nanofilm composition on a substrate (e.g. device, surface, composition, etc.), the substrate is then positioned in a desired environment (e.g. on a subject, within a subjects body, etc.), and the elution layer provides release (e.g. controlled release) of one or more compositions from within the intervening layer (e.g. layer between the base layer and elution layer (e.g. therapeutic layer)).

### Matrix Carrier/Delivery Element

In some embodiments, a carrier/delivery element is a matrix (e.g. a substantially crosslinked system). In some embodiments, a matrix is a three-dimensional crosslinked network. In some embodiments, an internal network structure within the matrix results from physical bonds, chemical bonds, crystallites, and/or other junctions. In some embodiments, a matrix is a substantially dilute crosslinked system. In some embodiments, a matrix comprises fluid within a three-dimensional crosslinked network. In some embodiments, a matrix comprises greater than 50% (e.g. >50%, >60%, >70%, >80%, >90%, >95%, >99%) fluid (e.g. water). In some embodiments, a matrix exhibits solid and/or liquid characteristics. In some embodiments, a matrix comprises a three-dimensional crosslinked network within a liquid (e.g. water). In some embodiments, a solid three-dimensional network spans the volume of a liquid medium. In some embodiments, a matrix comprises a hydrogel (aka aquagel). In some embodiments, a hydrogel comprises a network of polymer chains that are water-insoluble (e.g. colloidal gel) in which water is the dispersion medium. In some embodiments, a hydrogel comprises a network of water soluble polymer chains. In some embodiments, hydrogels may vary in strength, permeability, flexibility, hydration, pH, hardness, stickiness, etc. Methods and compositions for hydrogel preparation and use are well known in the art (U.S. Pat. No. 7,511,083 to Madsen, U.S. Pat. No. 7,413,752 to Sawhney, U.S. Pat. No. 7,407,912 Mertens, U.S. Pat. No. 7,329,414 to Fisher, U.S. Pat. No. 7,312,301 to Fang, U.S. Pat. No. 3,640,741 to Etes, U.S. Pat. No. 3,865,108 to Hartop, U.S. Pat. No. 3,992,562 to Denzinger et al., U.S. Pat. No. 4,002,173 to Manning et al., U.S. Pat. No. 4,014,335 to Arnold, U.S. Pat. No. 4,207,893 to Michaels, and in Handbook of Common Polymers, (Scott and Roff, Eds.) Chemical Rubber Company, Cleveland, Ohio.

In some embodiments, an additive element (e.g. therapeutic element) is embedded, contained, absorbed, and/or located within the matrix element (e.g. hydrogel). In some embodiments, a therapeutic element resides within a hydrogel element. In some embodiments, a therapeutic element is dissolved in a solvent (e.g. water) which provides the fluid portion of a matrix composition (e.g. hydrogel). In some embodiments, a matrix (e.g. hydrogel) provides a reservoir for loading a therapeutic element (e.g. drug, small molecule, macromolecule, etc.). In some embodiments, matrix characteristics (e.g. pore size, hydration level, matrix density, matrix composition, etc.) are tailored to provide a suitable/preferable carrier environment for a particular therapeutic element. In some embodiments, matrix characteristics (e.g. pore size, hydration level, matrix density, matrix composition, etc.) are tailored to provide a suitable/preferable delivery environment for a particular therapeutic element. In some embodiments, a matrix (e.g. hydrogel) provides release (e.g. controlled release) of a therapeutic element into the surrounding environment (e.g. biological system). In some embodiments, a therapeutic element elutes by diffusion from a matrix into the surrounding environment. In some embodiments, degradation of a matrix results in elution of a therapeutic element from within the matrix into the surrounding environment.

### B. Therapeutic Element

### Therapeutics

In some embodiments, the present invention provides a therapeutic element for the treatment and/or prevention of a disease, disorder, discomfort, ailment, etc. In some embodiments, the present invention provides compositions, devices, materials, methods, etc. for the release (e.g. controlled release) of a therapeutic element (e.g. into the surrounding environment). In some embodiments, a therapeutic element is embedded, encapsulated, contained, or layered in a carrier/delivery element. In some embodiments, a therapeutic element elutes from a carrier/delivery element. In some embodiments, a therapeutic element elutes from a carrier/delivery element at a desired or designed time scale (e.g. approximately 1 second, approximately 5 seconds, approximately 10 seconds, approximately 30 seconds, approximately 1 minute, approximately 5 minutes, approximately 10 minutes, approximately 30 minutes, approximately 1 hour, approximately 2 hours, approximately 4 hours, approximately 12 hours, approximately 24 hours, approximately 2 days, approximately 4 days, approximately 1 week, approximately 1 month, approximately 1 year, greater than 1 year, etc.). In some embodiments, a therapeutic element elutes from a carrier/delivery element with a desired or designed half life (e.g. approximately 1 second, approximately 5 seconds, approximately 10 seconds, approximately 30 seconds, approximately 1 minute, approximately 5 minutes, approximately 10 minutes, approximately 30 minutes, approximately 1 hour, approximately 2 hours, approximately 4 hours, approximately 12 hours, approximately 24 hours, approximately 2 days, approximately 4 days, approximately 1 week, approximately 1 month, approximately 1 year, greater than 1 year, etc.). In some embodiments, a therapeutic element elutes through pores and/or pinholes in a carrier/delivery element. In some embodiments, a therapeutic element elutes upon degradation and/or dissolving of a carrier/delivery element in or on the surrounding environment (e.g. body fluid, body surface, etc.).

In some embodiments, the present invention provides a therapeutic element comprising one or more therapeutics from the list including, but not limited to thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, gene therapy agents, etc.

In some embodiments, a therapeutic element comprises one or more drugs from one or more of the following classes:, 5-alpha-reductase inhibitors, 5-aminosalicylates, 5HT3 receptor antagonists, adamantane antivirals, adrenal cortical steroids, adrenergic bronchodilators, agents for hypertensive emergencies, agents for pulmonary hypertension, aldosterone receptor antagonists, alkylating agents, alpha-glucosidase inhibitors, alternative medicines, amebicides, aminoglycosides, aminopenicillins, aminosalicylates, amylin analogs, analgesic combinations, analgesics, androgens and anabolic steroids, angiotensin converting enzyme inhibitors, angiotensin II inhibitors, anorectal preparations, anorexiants, antacids, anthelmintics, anti-angiogenic ophthalmic agents, anti-infectives, antiadrenergic agents, centrally acting, antiadrenergic agents, peripherally acting, antianginal agents, antiarrhythmic agents, antiasthmatic combinations, antibiotics/antineoplastics, anticholinergic antiemetics, anticholinergic antiparkinson agents, anticholinergic bronchodilators, anticholinergics/antispasmodics, anticoagulants, anticonvulsants, antidepressants, antidiabetic agents, antidiabetic combinations, antidiarrheals, antidotes, antiemetic/antivertigo agents, antifungals, antigout agents, antihistamines, antihyperlipidemic agents, antihyperlipidemic combinations, antihypertensive combinations, antihyperuricemic agents, antimalarial agents, antimalarial combinations, antimalarial quinolines, antimetabolites, antimigraine agents, antineoplastic detoxifying agents, antineoplastic interferons, antineoplastic onoclonal antibodies, antineoplastics, antiparkinson agents, antiplatelet agents, antipseudomonal penicillins, antipsoriatics, antipsychotics, antirheumatics, antiseptic and germicides, antitoxins and antivenins, antituberculosis agents, antituberculosis combinations, antitussives, antiviral agents, antiviral combinations, antiviral interferons, anxiolytics, sedatives, and hypnotics, atypical antipsychotics, azole antifungals, bacterial vaccines, barbiturate anticonvulsants, barbiturates, benzodiazepine anticonvulsants, benzodiazepines, beta-adrenergic blocking agents, beta-lactamase inhibitors, bile acid sequestrants, biologicals, bisphosphonates, bronchodilator combinations, bronchodilators, calcium channel blocking agents, carbamate anticonvulsants, carbapenems, carbonic anhydrase inhibitor anticonvulsants, carbonic anhydrase inhibitors, cardiac stressing agents, cardioselective beta blockers, cardiovascular agents, central nervous system agents, cephalosporins, cerumenolytics, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, dibenzazepine anticonvulsants, digestive enzymes, dipeptidyl peptidase 4 inhibitors, diuretics, dopaminergic antiparkinsonism agents, drugs used in alcohol dependence, echinocandins, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycoprotein platelet inhibitors, glycylcyclines, gonadotropin releasing hormones, gonadotropins, group I antiarrhythmics, group II antiarrhythmics, group III antiarrhythmics, group IV antiarrhythmics, group V antiarrhythmics, growth hormone receptor blockers, growth hormones, H. pylori eradication agents, H2 antagonists, hematopoietic stem cell mobilizer, heparin antagonists, heparins, herbal products, hormone replacement therapy, hormones, hormones/antineoplastics, hydantoin anticonvulsants, illicit (street) drugs, immune globulins, immunologic agents, immunosuppressive agents, impotence agents, in vivo diagnostic biologicals, incretin mimetics, inhaled corticosteroids, inotropic agents, insulin, insulin-like growth factor, integrase strand transfer inhibitor, interferons, intravenous nutritional products, iodinated contrast media, ionic iodinated contrast media, iron products, ketolides, laxatives, leprostatics, leukotriene modifiers, lincomycin derivatives, local injectable anesthetics, loop diuretics, lung surfactants, lymphatic staining agents, lysosomal enzymes, macrolide derivatives, macrolides, magnetic resonance imaging contrast media, MAO Inhibitors, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous biologicals, miscellaneous cardiovascular agents, miscellaneous central nervous system agents, miscellaneous coagulation modifiers, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoclonal antibodies, mouth and throat products, mTOR kinase inhibitors, mucolytics, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neuraminidase inhibitors, neuromuscular blocking agents, next generation cephalosporins, nicotinic acid derivatives, NNRTIs, non-cardioselective beta blockers, non-iodinated contrast media, non-ionic iodinated contrast media, non-sulfonylureas, nonsteroidal anti-inflammatory agents, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, otic anesthetics, otic anti-infectives, otic preparations, otic steroids, otic steroids with anti-infectives, oxazolidinedione anticonvulsants, penicillinase resistant penicillins, penicillins, peripheral opioid receptor antagonists, peripheral vasodilators, peripherally acting antiobesity agents, phenothiazine antiemetics, phenothiazine antipsychotics, phenylpiperazine antidepressants, plasma expanders, platelet aggregation inhibitors, platelet-stimulating agents, polyenes, potassium-sparing diuretics, probiotics, progestins, prolactin inhibitors, protease inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, serotoninergic neuroenteric modulators, sex hormone combinations, sex hormones, skeletal muscle relaxant combinations, skeletal muscle relaxants, smoking cessation agents, spermicides, SSNRI antidepressants, SSRI antidepressants, Statins, sterile irrigating solutions, streptomyces derivatives, succinimide anticonvulsants, sulfonamides, sulfonylureas, tetracyclic antidepressants, tetracyclines, therapeutic radiopharmaceuticals, thiazide diuretics, thiazolidinediones, thioxanthenes, third generation cephalosporins, thrombin inhibitors, thrombolytics, thyroid drugs, topical acne agents, topical agents, topical anesthetics, topical anti-infectives, topical antibiotics, topical antifungals, topical antihistamines, topical antipsoriatics, topical antivirals, topical astringents, topical debriding agents, topical depigmenting agents, topical emollients, topical steroids, topical steroids with anti-infectives, toxoids, triazine anticonvulsants, tricyclic antidepressants, tumor necrosis factor (TNF) inhibitors, kinase inhibitors, ultrasound contrast media, upper respiratory combinations, urea anticonvulsants, urinary anti-infectives, urinary antispasmodics, urinary pH modifiers, vaginal anti-infectives, preparations, vasodilators, vasopressin antagonists, vasopressors, viral vaccines, viscosupplementation agents, vitamin and mineral combinations, vitamins, etc.

In some embodiments, a therapeutic element comprises a pharmaceutically acceptable carrier. In some embodiments, a therapeutic element is administered via any desired oral, parenateral, topical, intervenous, transmucosal, and/or inhalation routes. In some embodiments, a therapeutic element comprises a composition which is formulated with a pharmaceutically acceptable carrier and optional excipients, flavors, adjuvants, etc. in accordance with good pharmaceutical practice. In some embodiments, the present invention may be in the form of a solid, semi-solid or liquid dosage form: such as patch, tablet, capsule, pill, powder, suppository, solution, elixir, syrup, suspension, cream, lozenge, paste, spray, etc. As those skilled in the art would recognize, depending on the chosen route of administration, the composition form is determined. In general, it is preferred to use a unit dosage form of the therapeutic element in order to achieve an easy and accurate administration of the active compound. In general, the therapeutically effective pharmaceutical compound is present in such a dosage form at a concentration level ranging from about 0.5% to about 99% by weight of the total therapeutic element: i.e., in an amount sufficient to provide the desired unit dose. In some embodiments, the therapeutic element may be administered in single or multiple doses. The particular route of administration and the dosage regimen will be determined by one of skill in keeping with the condition of the individual to be treated and said individual's response to the treatment. The present invention also provides a therapeutic element in a unit dosage form for administration to a subject, comprising a pharmaceutical compound and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions. Suppositories for rectal administration of the pharmaceutical compound can be prepared by mixing the therapeutic with a suitable nonirritating excipient such as cocoa butter and polyethylene glycols, which are solid at ordinary temperatures but liquid at body temperature and which therefore melt in the rectum and release the drug. Additionally, it is also possible to administer the aforesaid pharmaceutical compounds topically and this may be preferably done by way of patch, cream, salve, jelly, paste, ointment and the like, in accordance with the standard pharmaceutical practice.

### Nanodiamond Functionalization

In some embodiments, a therapeutic element of the present invention comprises one or more nanodiamonds, nanodiamond clusters, nanodiamond film, functionalized nanodiamonds, functionalized-nanodiamond film, and/or functionalized-nanodiamond clusters. In some embodiments, any suitable therapeutic compound (e.g. drug, small molecule, macromolecule, etc.) including, but not limited to those listed herein, is provided in conjunction with one or more nanodiamond complexes in the therapeutic element of the present invention.

Nanodiamonds with diameters of approximately 2-8 nm are assembled into closely packed ND complexes (e.g. multilayer ND nanofilm, ND nanoclusters, etc.). In some embodiments, ND nanofilms are of any suitable thickness (e.g. 2 nm . . . 5 nm . . . 10 nm . . . 20 nm . . . 50 nm . . . 100 nm . . . 200 nm, etc.). In some embodiments, nanoclusters are of any suitable diameter (e.g. (e.g. 5 nm . . . 10 nm . . . 20 nm . . . 50 nm . . . 100 nm . . . 200 nm, etc.). In some embodiments, ND nanofilms and/or ND nanoclusters comprise multiple nanodiamonds. In some embodiments, ND nanofilms and/or ND nanoclusters of the present invention comprise integrated therapeutic compounds and/or complexes. In some embodiments, therapeutic compounds and/or complexes are embedded within ND nanofilms and/or ND nanoclusters. In some embodiments, the structures of ND nanofilms and/or ND nanoclusters prepared by methods of the present invention are adjusted according to the desired application (e.g. size, thickness, diameter, ND:therapeutic ratio, type of therapeutic, etc.). In some embodiments, functionalized ND nanocomplexes (e.g. nanofilms, nanoclusters, etc.) comprise ND nanocomplexes integrated with therapeutic molecules. In some embodiments, functionalized ND nanocomplexes are configured to provide controlled release of therapeutic molecules into or onto the surrounding environment (e.g. body fluid, body surface, etc.). In some embodiments, a therapeutic element comprises one or more ND nanocomplexes combined with additional elements, compositions, materials, compounds, complexes, carriers, additives, etc.

### C. Applications

In some embodiments, one or more carrier/delivery elements and one or more therapeutic elements of the present invention are combined to provide a material, composition, and/or device of the present invention. Any combination of embodiments of the carrier/delivery elements and therapeutic elements described explicitly or inherently herein are contemplated. In some embodiments, the present invention provides a coating for devices, surfaces, substrates, compositions, materials, etc. In some embodiments, devices, surfaces, substrates, compositions, or materials coated according to the present invention are configured to administer one or more therapeutic compositions. In some embodiments the present invention is applied to medical devices such as a balloon catheter, an atherectomy catheter, a drug delivery catheter, a stent delivery catheter, an endoscope, an introducer sheath, a fluid delivery device, other infusion or aspiration devices, device delivery (i.e. implantation) devices, and the like.

In some embodiments, the present invention provides a medical device with one or more of its surfaces coated with a composition, material, or nanofilm described herein. The medical device may be implantable. In some embodiments the medical device contains an electrode. A coating of the present invention may be used on a variety of medical substrates, including an implantable medical device. Such medical devices may be made of a variety of biocompatible materials including, but not limited to, any suitable metal or non-metal material (e.g. metals (e.g. Lithium, Magnesium, Aluminium, Titanium, Vanadium, Chromium, Manganese, Cobalt, Nickel, Copper, Zinc, Zirconium, Molybdenium, Silver, Cadmium, Antimony, Barium, Osmium, Platinum, Mercury, Thallium, Lead, etc.), plastics (e.g. Bakelite, neoprene, nylon, PVC, polystyrene, polyacrylonitrile, PVB, silicone, rubber, polyamide, synthetic rubber, vulcanized rubber, acrylic, polyethylene, polypropylene, polyethylene terephthalate, polytetrafluoroethylene, gore-tex, polycarbonate, etc.), etc. Medical substrates onto which the composition and/or materials of the present invention are coated include, neural/cardiovascular/retinal implants, leads and stents, and dental implants (e.g., nanofilms to seed bone growth). In some embodiments, the materials, compositions and/or nanofilm may be coated onto the electrode of an implantable medical device. In fact, coating the present materials, compositions and/or nanofilms onto an electrode is contemplated to provide important medical advantage because the materials, compositions and/or nanofilm is contemplated to prevent or minimize bio-fouling which often begins at the site of a metal electrode. In addition, unlike more conventional implant coatings, the present coatings may be made thin enough that they do not interfere with electrode function (e.g., electrical conductivity or redox reactions at electrodes). Other medical device uses and configurations will be understood by one skilled in the art using the principles described herein.

### D. Selected Embodiments

### Parylene Bilayer

The following provides embodiments of the present invention in which a therapeutic layer is embedded between two or more Parylene layers, such as a base layer and an elution layer. The embodiments herein should not be construed as limiting the scope of the invention, and may be utilized in combination with any other embodiments contemplated and/or disclosed throughout the present application.

Certain members of the poly-p-xylenes family, commonly known as Parylene, have been used as coatings for medically implanted devices due to their biocompatibility (Eskin et al. Journal of Biomedical Materials Research, 1976, 10, 113., Fontaine et al. 1996, 3, 276., herein incorporated by reference in their entireties) (USP approved Class VI polymer). The nature of the chemical deposition process (CVD), through which the polymerization of Parylene takes place, allows for the formation of a conformal barrier (Fortin & Lu. Chemical Vapor Deposition Polymerization The Growth and Properties of Parylene Thin Films; Kluwear: Norwell, 2004., herein incorporated by reference in its entirety) between the medical device and exterior environment. Another advantage to the deposition process is that it occurs at room temperature (Dolbier & Beach. Journal of Fluorine Chemistry 2003, 122, 97., herein incorporated by reference in its entirety), preserving device function. Applications of Parylene derivatives such as Parylene C (dichloro(2,2)paracyclophane) and Parylene N ((2,2)paracyclophane) include a range of devices from catheters (Bruck. Blood Compatible Synthetic Polymers CC Thomas: Springfield, 1974, herein incorporated by reference in its entirety) to stents. Recent efforts to supplement the integration of Parylene coated medical devices by applying alternative drug-conjugated polymeric coatings to the Parylene surface, have been successful in the fabrication of drug eluting stents (Westedt et al. Journal of Controlled Release 2006, 111, 235., Unger et al. Journal of Controlled Release 2007, 117, 312., herein incorporated by reference in their entireties). These multi-polymeric coatings complement the biomaterial platform. Developing biocompatible polymeric coatings with a greater degree of biological activity has been a struggle in the field. Experiments were conducted during the development of embodiments of the invention to assess the drug eluting potential of an amine functionalized poly-p-xylene, known as Parylene A (4-amino(2,2) paracyclophane). Murine macrophage cell line RAW 264.7 served as a cellular response to dexamethasone (DEX), a synthetic anti-inflammatory gluco-corticoid, and doxorubicin, an anticancer therapeutic. DEX was used as an example of a therapeutic that can be used with a Parylene A nanofilm. Decrease of NFK-B mediated cytokines, Interleukin-6 (IL-6) and Tumor Necrosis Factor-α (TNF-α), resultant DNA fragmentation, and spectroscopic analysis revealed the drug eluting properties of a Parylene A polymeric bilayer. These experiments demonstrated that Parylene A finds use in drug delivery devices.

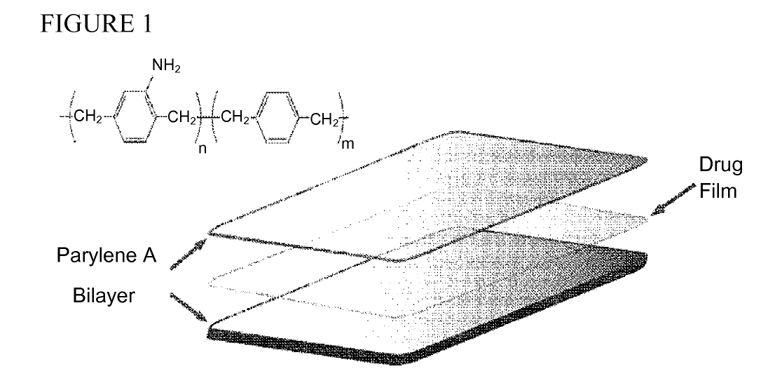

For example, experiments were conducted during the development of embodiments of the invention, to expand upon the capability of differentially functionalized poly-p-xylene (Parylene) derivatives, and to examine the drug eluting potential of Parylene A. An exemplary deposition of Parylene A occurred in a two step fashion wherein a coating of dexamethasone (DEX) (anti inflammatory agent) or doxorubicin (DOX) (anti-cancer therapeutic), used as examples of useful therapeutic agents, was dispersed between a primary base layer and a secondary elution layer (SEE FIG. 1). The polymerization reaction of the secondary layer allowed for the elution of therapeutic agents to be examined. The release of the underlying drug was accomplished by restricting the amount of polymer (e.g., Parylene A) available for the deposition process, and depriving the polymerization reaction sufficient material to coat the surface in a conformal manner; pinholes formed as a result, although the present invention is not limited to any particular mechanism of action and an understanding of the mechanism of action is not necessary to practice the present invention. Prior to experiments conducted during the development of embodiments of the invention, an investigation concerning the surface characterization of micro to nanoscale deposition was incomplete; however, reports available revealed the presence of pinholes at or below the micron level (Spellman et al. 1999, 15, 308., herein incorporated by reference in its entirety). Previous work also indicated the elution of underlying material from a nanoscale deposition of Parylene C (Zeng et al. Biomacromolecules 2005, 6, 1484., herein incorporated by reference in its entirety). Experiments were conducted during the development of embodiments of the invention to characterize the disposition, frequency, size, and concentration of pinhole formation within micro to nanoscale Parylene A films. The control over film properties provided by the present invention permits optimization of drug elution over time in a site-specific concentration-dependent fashion.

The application of two different classes of drugs, dexamethasone, an anti-inflammatory and doxorubicin, a chemotherapeutic, revealed an exemplary range of medicinal agents which can be incorporated into a Parylene A polymeric device, although medical agents outside this range may be utilized with the present invention. Experiments conducted during the development of embodiments of the invention demonstrate that drug concentration attained levels comparable to their respective controls, and drug function was retained following the application of the secondary eluting layer. It is contemplated that this platform finds use to present a biocompatible surface with adherent biomolecules (e.g., proteins (De Bartolo et al. Biomolecular Engineering 2007, 24, 23., Lopez et al. Surface and Coatings Technology 2005, 200, 1000., herein incorporated by reference in their entireties) or other biological agents (Hoffman. Clinical Chemistry 2000, 46, 1478., herein incorporated by reference in its entirety), covalently linked to surface amine groups, while maintaining site specific drug eluting properties. A wide range of desired biomolecules or other molecules may be conjugated to the amine groups to provide desired properties to the device.

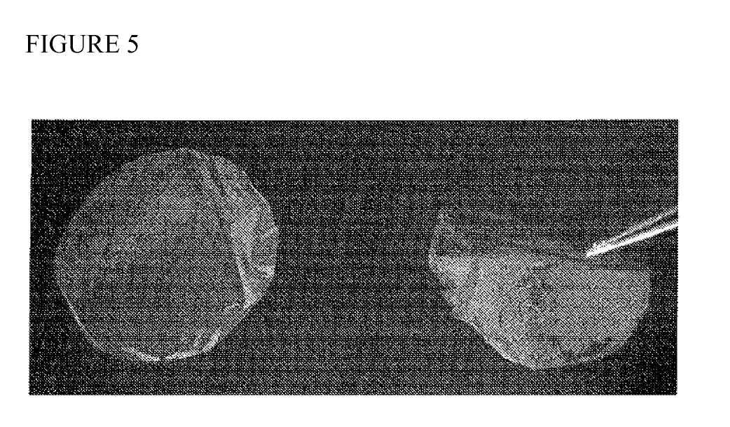

A Parylene A nanofilm comprising a therapeutic patch (SEE FIG. 5), or providing a coating for a biomedical device, augments existing medical treatments in a non-invasive fashion. Incorporation of an immunosuppressant such as DEX into the coating of a medical device inhibits localized inflammation at the source of implantation, reducing scarring and expediting recovery time. In some embodiments, application of an anti-cancer eluting device, utilizing a chemotherapeutic-eluting Parylene A nanofilm, provides localized delivery of chemotherapeutic agents following post-surgical tumor excision decreasing the incidence of tumor resurgence. Other therapeutic uses and configurations will be understood by one skilled in the art using the principles described herein.

### Therapeutic-Functionalized Nanodiamonds Within a Bilayer

The following provides embodiments of the present invention in which therapeutic-functionalized nanodiamonds are embedded within the layers of a bilayer, such as a base layer and an elution layer. The embodiments herein should not be construed as limiting the scope of the invention, and may be utilized in combination with any other embodiments contemplated and/or disclosed throughout the present application.

Numerous synthetic and natural nanoscale carriers have been developed and investigated for modulating therapeutic release. These include, but are not limited to polymer-protein conjugates, liposomes, micelles, dendrimers, polyelectrolyte films, co-polypeptides, carbon nanotubes, and variants of the above (Peer et al. Nature Nanotechnology, 2007, 18, 751., Volodkin et al. Soft Matter; 2008, 4, 122., Langer. Science, 1990, 249, 1527., Wood et al. Proceedings National Academy of Sciences, 2006, 103, 10207., Wood et al. Langmuir, 2005, 21, 1603., Deming. Advanced Drug Delivery Reviews, 2002, 54, 1145., Lacerda et al. Advanced Drug Delivery Review, 2006, 58, 1460., herein incorporated by reference in their entireties). Nanodiamonds (NDs) in particular possess several characteristics that make them suitable for advanced drug delivery. NDs with individual diameters of 2-8 nm have been functionalized with doxorubicin (DOX) (Huang et al. Nano Letters, 2007, 7, 3305., herein incorporated by reference in its entirety). Due to their high surface to volume ratio and non-invasive dimensions, extremely high loading capacities of therapeutic were achieved. NDs possess tailorable surface properties and compositions providing the capability to interface with virtually any therapeutic molecule via physical interactions (Huang et al. ACS Nano, 2008, 2, 203, herein incorporated by reference in its entirety). With highly ordered aspect ratios near unity, NDs have been shown to be biologically stable, allowing them to preclude adverse cellular stress and inflammatory reactions. Several reports have confirmed the inherently amenable biological performance of suspended NDs when interacting with cells (Huang et al. Nano Letters, 2007, 7, 3305., Huang et al. ACS Nano, 2008, 2, 203., Schrand et al. The Journal of Physical Chemistry Letters, 2007, 111, 2., Liu et al. Nanotechnology, 2007, 18, 325102., Yu et al. Journal of American Chemical Society, 2005, 127, 17604, herein incorporated by reference in their entireties). The general cellular viability, morphology and mitochondrial membrane is maintained amongst various cell types when incubated with suspended NDs. As such, with appropriate manipulation of drug elution parameters in conjunction with proper selection of material matrices, NDs serve as promising platforms for sustained and localized therapeutic release.

Previous studies functionalizing ND films fabricated via chemical vapor deposition (CVD) with various biological entities have provided exciting prospects for biosensing applications but are nonetheless difficult to implant due to their rigidity (Hartl et al. Nature Materials, 2004, 3, 736., Yang et al. Nature Materials, 2002, 1, 253, herein incorporated by reference in their entireties). Experiments were conducted during the development of embodiments of the present invention to develop an easily fabricated flexible nanofilm device capable of extended localized chemotherapeutic drug delivery. It is contemplated that through targeted slow release, continuously administered small dosages replicate the efficacy of normally larger prescribed amounts, reducing side effects generally associated with systemic chemotherapeutic treatments (Lankelma et al. Clinical Cancer Research, 1999, 5, 1703., Legha et al. Annals of Internal Medicine, 1982, 96, 133., Legha et al. Cancer, 1982, 49, 1762., herein incorporated by reference in their entireties).

Parylene C, a material with well-documented biocompatibility and FDA-approval, was used as an example of a useful, flexible, and robust framework for the path (Hahn et al. Journal of Applied Polymer Science Applied Polymer Symposium, 1984, 38, 55., Yamagishi. Thin Solid Films (Switzerland), 1991, 202, 39., herein incorporated by reference in their entireties). Parylene coatings have been utilized in several medical applications due to their highly conformal nature, biostability and inertness under physiological conditions with no known biological degradation events.

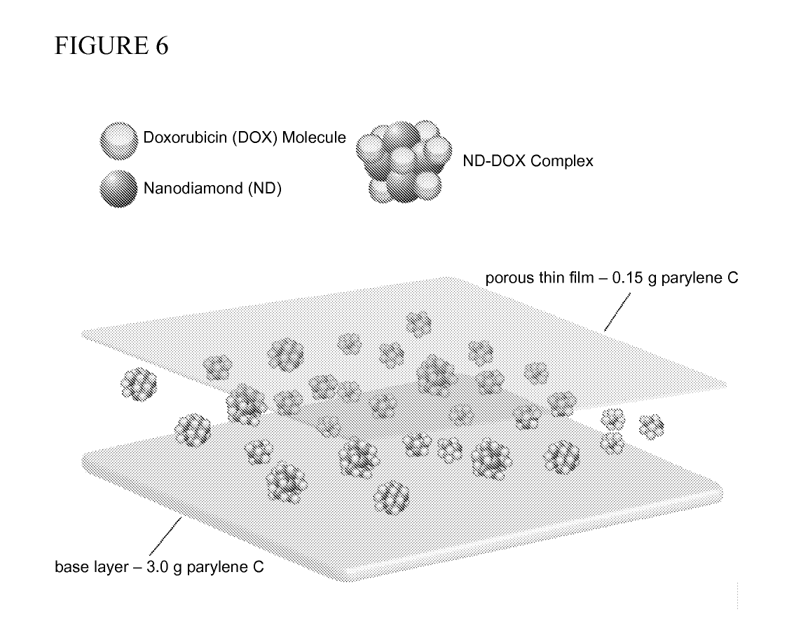

An exemplary hybrid film was made and tested. The hybrid film comprised DOX-functionalized NDs, sandwiched between a thick hermetic base and thin permeable layer of Parylene C (SEE FIG. 6A). NDs efficiently sequestered DOX, and could be released gradually upon appropriate stimuli (e.g. DOX concentration gradients and acidic pH conditions, which have been shown to be indicative of cancerous cells); although the present invention is not limited to any particular mechanism of action and an understanding of the mechanism of action is not necessary to practice the present invention. A permeable top layer of Parylene C acted as an additional physical barrier that further limits and modulates elution. NDs have previously been functionalized with cytochrome c, DNA, and various protein antigens (Huang & Chang, Langmuir, 2004, 20, 5879., Vshizawa et al. Chemical Physics Letters, 2002, 351, 105., Kossovsky et al. Bioconjugate Chemistry, 1995, 6, 507., herein incorporated by reference in their entireties). Experiments have previously demonstrated the ability to functionalize NDs with the apoptosis-inducing chemotherapeutic agent, DOX, and anti-inflammatory immunosuppressant glucocorticoid, dexamethasone. However, to enhance the applicability of the NDs as foundational elements for device fabrication, experiments were conducted during the development of embodiments of the invention to generate a localized elution device to address a broad range of medical conditions including difficulties involving tumor heterogeneity, blood circulation and unsustainable controlled release over a prolonged period of time (Jain, Advanced Drug Delivery Reviews, 2001, 46, 149., herein incorporated by reference in its entirety). Therefore, the ND-Parylene hybrids possess particular significance and relevance towards oncological and anti-inflammatory translation. Furthermore, the biocompatible properties, customization, and localization capacity of the flexible Parylene C encapsulated DOX-ND hybrid film (SEE FIG. 6B-C), of the present invention, addressed several of these difficulties and provides a method for a wide variety of drug delivery schemes.

Hybrid polymer-ND based films were constructed as a flexible, robust and slow drug release device useful as implants or as stand-alone devices for specific therapies such as antitumor patches. This device configuration provides a platform on which a wide variety of therapeutic drug delivery devices could be developed. These devices also find use in research settings. In some embodiments, two or more therapeutics agents are provided in a device and/or two or more devices each having one agent are utilized. Hybrid films were capable of releasing a continuous amount of drug for at least a month. By altering drug-ND deposition amounts and the thickness of the permeable Parylene layer, dosage amounts and thus, total release times can be calibrated. Since drug release is presumably driven by drug concentration gradients, it is contemplated that the devices can be optimized to reduce elution rates, should the local therapeutic concentration reach a defined threshold. The flexibility of the biocompatible structural material (e.g. Parylene) and the drug sequestering element, NDs, provide an invention with numerous uses involving adjustable and extended timed release with a variety of therapeutics.

### Therapeutic-Functionalized Nanodiamonds Within Hydrogel Matrix

The following provides embodiments of the present invention in which therapeutic-functionalized nanodiamonds are embedded within a hydrogel matrix. The embodiments herein should not be construed as limiting the scope of the invention, and may be utilized in combination with any other embodiments contemplated and/or disclosed throughout the present application.

Nanodiamonds (NDs) contain several unique features beneficial to potential biomedical applications. Due to the surface characteristics of the diamond surface, ND particles can be functionalized and bound to a variety of biological agents (Huang & Chang. Langmuir, vol. 20, pp. 5879-5884, 2004., Ushizawa et al. Chemical Physics Letters, vol. 351, pp. 105-108, 2002., Kossovsky et al. Bioconjugate Chemistry, vol. 6, pp. 507-511, September-October 1995., Kruger. Angewandte Chemie-International Edition, vol. 45, pp. 6426-6427, 2006., herein incorporated by reference in their entireties) implemented for the capture and separation of specific proteins, and used as fluorescent markers for cell imaging (Yeap et al. Analytical Chemistry, vol. 80, pp. 4659-4665, June 2008., Bondar et al. Physics of the Solid State, vol. 46, pp. 758-760, 2004., Yu et al. Journal of the American Chemical Society, vol. 127, pp. 17604-17605, December 2005., Neugart et al. Nano Letters, vol. 7, pp. 3588-3591, December 2007., Chang et al. Nature Nanotechnology, vol. 3, pp. 284-288, May 2008., herein incorporated by reference in their entireties). In addition, NDs find use in a variety of biocompatibility assays, including evaluation of viability, mitochondrial function, ATP production, and genetic profiles for inflammation (Schrand et al. Journal of Physical Chemistry B, vol. 111, pp. 2-7, 2007., Bakowicz & Mitura. Journal of Wide Bandgap Materials, vol. 9, p. 12, 2002., Liu et al. Nanotechnology, vol. 18, p. 10, August 2007., Huang et al. Nano Letters, vol. 7, pp. 3305-3314, 2007., herein incorporated by reference in their entireties). Developments in breaking up large particle aggregates have further contributed to the potential in applying NDs in biomedical practice (Ozawa et al. Advanced Materials, vol. 19, pp. 120, May 2007., Kruger et al. Carbon, vol. 43, pp. 1722-1730, July 2005., herein incorporated by reference in their entireties).

In addition to the high surface area-to-volume ratio, ND powders contain an abundant amount of defects on their surface, resulting in large surface areas up to 450 m² g-1 (Dolmatov. Uspekhi Khimii, vol. 70, pp. 687-708, 2001., herein incorporated by reference in its entirety). These large surface areas offer advantageous loading capacities for attaching therapeutics, which can be further improved with additional surface modification. Experiments performed during development of embodiments of the present invention impart unique nanoparticle features towards the macroscale by immobilizing NDs within a polymer matrix.

Widely used, poly(ethylene glycol) (PEG) hydrogels have been used to immobilize and release oligonucleotides, proteins, growth factors, drugs, enzymes and various cells (West & Hubbell. Reactive Polymers, vol. 25, pp. 139-147, 1995., Gayet & Fortier. Journal of Controlled Release, vol. 38, pp. 177-184, 1996., Scott & Peppas. Biomaterials, vol. 20, pp. 1371-1380, 1999., Andreopoulos et al. Biotechnology and Bioengineering, vol. 65, pp. 579-588, December 1999., Anseth et al. Journal of Controlled Release, vol. 78, pp. 199-209, 2002., Bhattacharjee et al. Journal of Nanoparticle Research, vol. 4, pp. 225-230, 2002., Gattas-Asfura et al. Journal of Physical Chemistry B, vol. 107, pp. 10464-10469, September 2003., herein incorporated by reference in their entireties). PEG has the desirable properties of biocompatibility, beneficial hydration, and adequate resistance towards protein adsorption and cell adhesion. Furthermore, as it is not easily recognized by the immune system, PEG reduces immunogenetic and antigenic reactions of proteins in vivo (Fuertges & Abuchowski. Journal of Controlled Release, vol. 11, pp. 139-148, 1990, herein incorporated by reference in its entirety). Versatility in controlling PEG concentrations due to innate water solubility and functional group modifications allow uncomplicated fundamental structural modifications towards obtaining desired release kinetics (Kim et al. Pharmaceutical Research, vol. 9, pp. 283-290, 1992, herein incorporated by reference in its entirety).

In some embodiments of the present invention, poly(ethylene glycol) diacrylate (PEGDA) with an average molecular weight of 575 was utilized within dental compounds and flexible coatings due to its chemical and impact resistance, flexibility, strength and adhesion (Sigma MSDS). In addition, PEGDA has proven biocompatibility, for which it has been implemented as a hydrogel crosslinker and scaffold in tissue applications (Zheng Shu et al. Biomaterials, vol. 25, pp. 1339-1348, 2004, herein incorporated by reference in its entirety).

A myriad of nanoparticle-hydrogel hybrids have been developed (Bhattacharjee et al. Journal of Nanoparticle Research, vol. 4, pp. 225-230, 2002., Gattas-Asfura et al. Journal of Physical Chemistry B, vol. 107, pp. 10464-10469, September 2003, Huang et al. Journal of Controlled Release, vol. 94, pp. 303-311, February 2004., herein incorporated by reference in their entireties). The immobilization of drug conjugated 2-8 nm diameter ND particles (Huang et al. Nano Letters, vol. 7, pp. 3305-3314, 2007., Huang et al. ACS Nano, vol. 2, pp. 203-212, 2008., herein incorporated by reference in their entireties) within a PEGDA polymer capable provided extended drug storage. In particular, NDs bound with the apoptosis-inducing chemotherapeutic, doxorubicin hydrochloride (DOX) demonstrated an easy and direct method of analyzing drug release due to its strong absorbance.

Experiments were performed during development of embodiments of the present invention in which ND:PEGDA hydrogels were demonstrated to concurrently sequester and slow-release drug while avoiding burst release effects. In addition, the gels were fabricated in an expeditious, economical and facile manner, providing high loading capacities without any additional complex steps. PEG in particular has several beneficial characteristics that make it suitable for clinical applications. ND:PEGDA hydrogels have demonstrated utility in biomedical applications, namely in coatings and tissue engineering.

Properties and release characteristics of ND:PEGDA hydrogels can be tailored by varying PEG molecular weight, crosslinking density, the swelling ratio, gel content or functional groups (Priola et al. Polymer, vol. 34, pp. 3653-3657, 1993., herein incorporated by reference in its entirety). In addition, the biocompatibility of the hydrogels can be further improved by altering photoinitiators without any loss in fabrication fidelity or simplicity (Bryant et al. Journal of Biomaterials Science-Polymer Edition, vol. 11, pp. 439-457, 2000., Williams et al. Biomaterials, vol. 26, pp. 1211-1218, 2005., herein incorporated by reference in their entireties).

In experiments performed during development of embodiments of the present invention, after two weeks of incubation, ND:PEGDA hydrogels did not release the bulk of the drug while standard hydrogels released virtually all of their initial reservoirs. Optimal release effects can be achieved by varying pH and degradation rates. In some embodiments, NDs are included with environmental gels that degrade within physiological conditions. In some embodiments, as the surrounding matrix is reduced, therapeutically modified NDs are released with the NDs serving as an antigen delivery vehicle. For example, anti-inflammatories can be adsorbed onto NDs and inserted into hydrogels as a contributive implant coating. For very specific location delivery, micropatterned hydrogels can be formed easily with existing micro-manufacturing techniques and non toxic solvents (Revzin et al. Langmuir, vol. 17, pp. 5440-5447, September 2001., Subramani & Birch. Biomedical Materials, vol. 1, pp. 144-154, September 2006., herein incorporated by reference in their entireties).

### EXPERIMENTAL

### Example 1

### Compositions and Methods for the Preparation and Characterization of Amine-Functionalized Parylene

Substrate Preparation. Plain glass slides were sterilized in 70% ethanol and pre-treated with A-174 Silane (SCS COATINGS, Indianapolis, Ind.) adhesion promoter according to manufacturer's protocol. Approximately 20 grams of Parylene A (UNIGLOBE KISCO, White Plains, N.Y.) was loaded into a Parylene deposition system (PDS) 2010 LABCOTER® 2 (SCS COATINGS.) The deposition took place under previously indicated conditions (Kramer et al. 1984, 22, 475., Yang et al. Journal of Crystal Growth 1998, 183, 385., herein incorporated by reference in their entireties). Application of drug to the base layer of Parylene A was accomplished via desiccation of 100 μg dexmnethasone (SIGMA ALDRICH, St Louis, Mo.), or 25 μg doxorubicin (U.S. PHARMACOPIA, Rockville, Md.) under a laminar flow hood. A second layer of 150 mg of Parylene A was deposited over the drug films to produce the eluting layer.

Cell Culture Conditions. Macrophage cell line RAW 264.7 (AICC Manassas, Va.) cells were grown in DMEM media (MEDIATECH Inc, Hemdon, Va.) supplemented with 10% FBS (AICC) and 1% Penicillin/Streptomycin (LONZA, Walkersville, Md.). Investigation of inflammation pathways utilized lipo-polysacchmide 5 ng/ml (SIGMA ALDRICH) and resultant expression of IL-6 and TNF-α genes. Analysis of cellular apoptosis was accomplished using agarose electrophoresis of DNA fragmentation.

Quantitative RT-PCR. RNA isolation was accomplished utilizing TRIZOL reagent (INVITROGEN Corporation, Carlsbad, Calif.) per manufacturer's guidelines. cDNA was synthesized using the ISCRIPT SELECT cDNA Synthesis Kit (BIO-RAD, Hercules, Calif.) PCR was done using SYBER Green detection reagents (QUANTA BIOSCIENCES, Gaithersburg, Md.) and appropriate primers for mIL-6, mTNF-α, and 3-Actin (INTEGRATED DNA TECHNOLOGIES, Coralville, Iowa). Samples were amplified using a MYIQ real-time PCR detection system (BIO-RAD).

Electrophoretic Assay. Cells were washed with PBS wash and removed from the substrate. Cells were lysed using a cell lysis solution (10 mM Tris-HCL, pH 8.0, 10 mM EDTA, 1% Triton X-100) and incubated with RNase and Protinase K. DNA was extracted using a 2% isoamyl alcohol (25:24:1) solution and precipitated in isopropanol. Remaining pellet was washed in 70% ethanol and re-suspended in DEPC water. DNA fragmentation was characterized via a 0.8% agarose gel using sodium borate buffer and ethidium bromide staining.

Spectroscopy Analysis. Microscope glass slides were cut to size and coated with a primary base layer of Parylene A as described previously. 0.25 mg of doxombicin (US PHARMACOPIA) was desiccated onto the individual glass sections and a secondary eluting layer of Parylene A (150 mg) was deposited. Samples were placed in a 12 well plate immersed in 1.0 ml of PBS for 10 min, 20 min, 40 min, 1 hour, 4 hour, and 24 hour increments. After each time point the solution was removed and the drug eluting glass disks were transferred to a vacant well and replenished with 10 ml of PBS. Remaining solutions were analyzed in a DU Series 700 UV-Vis Scanning Spectrophotometer (BECKMAN COULTER, Fullerton, Calif.).

### Example 2

### Characterization of Drug Elution from the Parylene A Bi-Layer

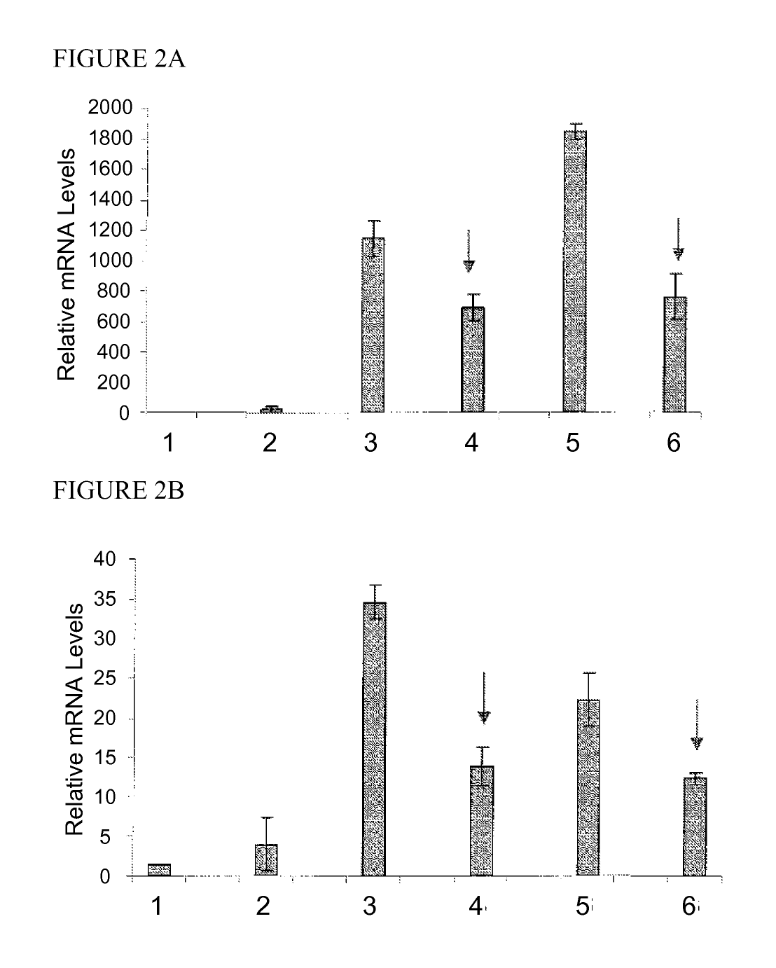

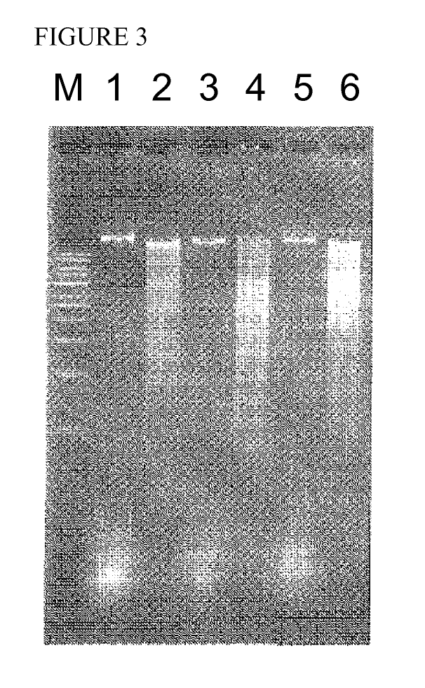

Characterization of drug elution from the Parylene A bi-layer was accomplished by comparing the activity of the eluted drug to the respective controls. In all tests, elution of the drug from controls and pinhole coated surfaces were equivalent as determined by the concentration and function of the released drug. This suggested that the secondary elution layer imposed no negative effect of upon drug function or elution. This was evident from the graphical representation of DEX mediated gene expression (SEE FIG. 2A-B), and of DOX mediated apoptosis, demonstrated by the onset of DNA fragmentation (SEE FIG. 3). The application of DEX has been shown to mediate downstream cytokines IL-6 and TNF-α (Jeong et al. 2003, 144, 4080., Maeda et al. Hearing Research 2005, 202, 154., herein incorporated by reference in their entireties). Lipopolysaccharide activation of the inflammatory cytokines IL-6 and TNF-α were decreased through the presence of DEX (SEE FIG. 2A-B).

Doxorubicin functions through the intercalation of DNA promoting cell mediated apoptosis (Jurisicova et al. Cell Death Differ, 2006, 13, 1466., Wang et al. Biochemical Journal 2002, 367, 729., Huang et al. Nano Letters 2007, 7, 3305., herein incorporated by reference in their entireties). Apoptotic behavior of RAW 264.7 cells was confirmed through the presence of laddering, indicative of cellular apoptosis, as noted in the electrophoretic separation of DNA (SEE FIG. 3). The amine functionalized surface had no negative effect on cell growth in regards to stimulating apoptotic pathways. The elimination of any detrimental growth effect can be asserted.

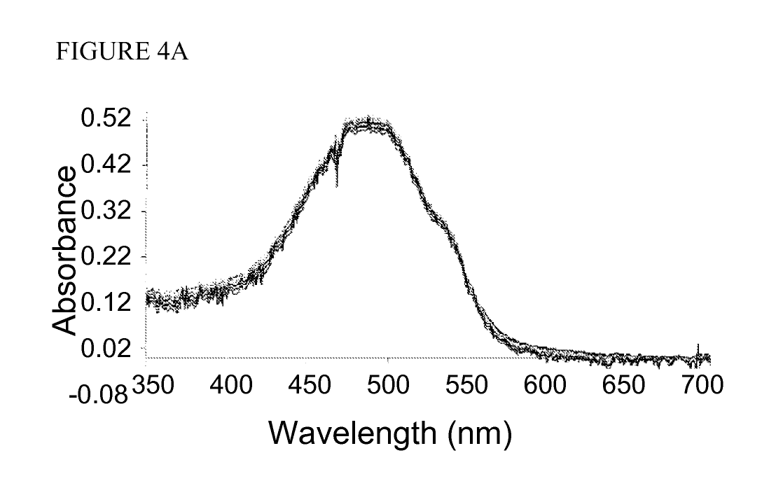

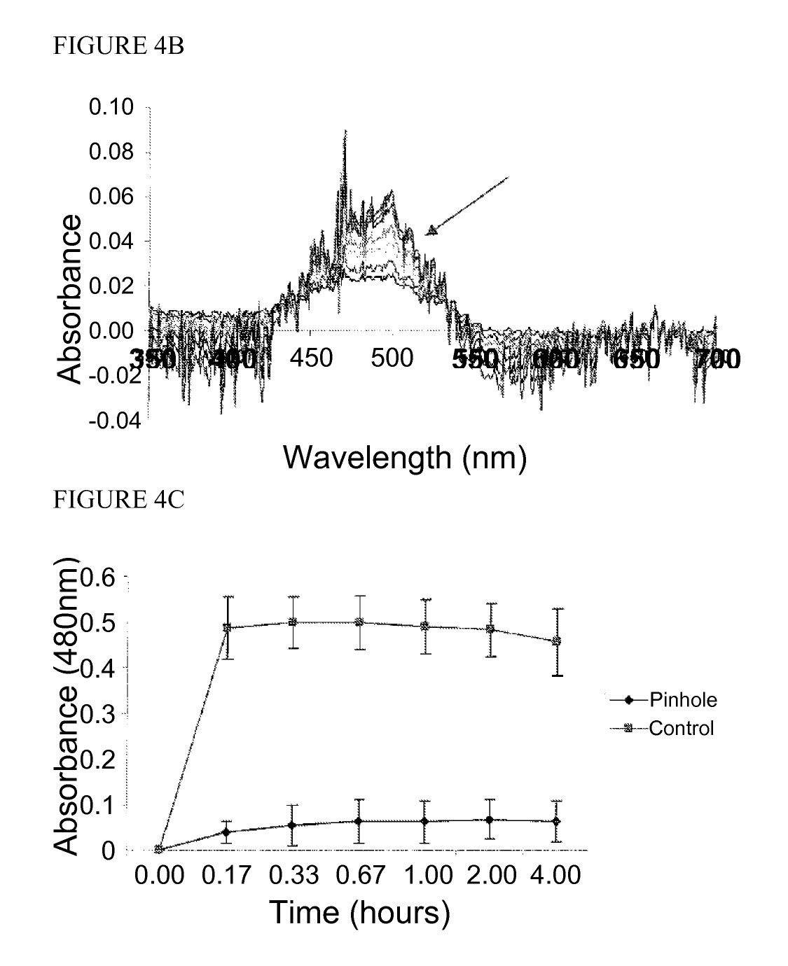

Spectroscopic analysis revealed the degree of DOX elution over time (SEE FIG. 4A-C). Compared to the control (SEE FIG. 4A), the gradual elution of DOX from alternating layers of Parylene A (SEE FIG. 4B) is evident. Further analysis integrating each sample was taken at the maximum absorbance of DOX at 480 nm over time (SEE FIG. 4C). Contrasting absorbance values provide further evidence to the porous nature of micro to nanoscale depositions of Parylene A. The experiments conducted during the development of embodiments of the invention affirmed the presence of physical pores, present within the superficial Parylene A layer, due to incomplete polymerization of the substrate surface.

It has been contemplated that the deposition of the non-adherent drug film to the underlying primary surface could result in shearing off of the secondary elution layer under applied mechanical forces. However, the experiments conducted during the development of embodiments of the invention, most notably the spectroscopic analysis (SEE FIG. 4A-C), support the notion of pinhole mediated release. Delamination of the secondary layer would have resulted in bi-layer elution rates comparable to that of the control, rather than the pinhole mediated release observed.

While the precise mechanism of pinhole formation has yet to be determined, it is clear that by introducing increasingly lower quantities of Parylene A into commercially available Parylene deposition systems results in the formation of porous films; although the present invention is not limited to any particular mechanism of action and an understanding of the mechanism of action is not necessary to practice the present invention. Experiments conducted during the development of embodiments of the invention were focused on porosity in regards to Parylene A. It is contemplated that deposition kinetics for other para-p-xylene derivatives may differ due to the range of functional groups and respective molecular masses.

### Example 3

Compositions and Methods for the Fabrication and Characterization of Nanodiamond-Embedded Devices

ND suspension and functionalization with DOX. NDs were functionalized and dispersed. Upon ND ultrasonication (100 W, VWR 150D sonicator) for 30 minutes, DOX and ND solutions were centrifuged together at appropriate concentrations at a 4:1 ratio. Addition of NaCl helped facilitate the process.

Materials and Device Fabrication. A conformal 3 g base layer of Parylene C was deposited on pre-cut 2.5 cm×2.5 cm glass slides with a SPECIALTY COATING SYSTEMS (SCS) PDS 2010 LABCOATER (SCS, Indianapolis, Ind.). The Parylene layer was oxidized via oxygen plasma treatment in a HARRICK Plasma Cleaner/Sterilizer (Ithaca, N.Y.) at 100 W for one minute. A DOX-ND solution was then added to the base layer so that the final DOX-ND concentration in solution was 6.6 μg/ml. Subsequently, solvent evaporation occurred in isolation at room temperature. Following DOX-ND deposition, an ultra-thin 0.15 g palylene C layer was deposited as an elution-limiting and control element. The final layer of Parylene C was treated with oxygen plasma at 100 W for one minute. Parylene C was pyrolized into a gaseous monomer at 690° C., and deposited at room temperature under vacuum conditions for all depositions. The preceding parameters apply to devices fabricated for spectroscopy studies. The present invention is not limited to these parameters. The concentration of DOX-ND was adjusted for devices used in the DNA fragmentation assay so that the final DOX-ND concentration in solution was 33 μg/ml.

Atomic Force Microscopy (AFM) Characterization. ASYLUM MFP3D AFM (Santa Barbara, Calif.) images of the samples were taken to identify the structure and interaction between proteins. Image dimensions were 20 μm×20 μm. Contact mode imaging at line scan rates of 03 to 0.5 Hz were performed in at room temperature with OLMPUS TR800PSA 200 μm length silicon nitride cantilevers (Melville, N.Y.).

Spectroscopic Analysis. Samples were immersed in 2 mL of nanopure water in 6-well plates and placed in an incubator at 37° C. and 5% CO₂. At every 24-hour interval, samples were transferred to another well to avoid residue contamination while the remaining 2 mL of eluate was collected. A full wavelength scan from 350 nm-700 nn was performed on 100 μl of the eluate with a BECKMAN COULTER DU730 Life Science UVI Vis Spectrophotometer (Fullerton, Calif.).

Contact Angle Measurements. Static contact angles were measured with 10 μl of DI water with a RAME-HART, Inc. Imaging System and Auto Pipetting System (Mountain Lakes, N.J.).

DNA Fragmentation Assay. RAW 264.7 murine macrophages (ATCC, Manassas, Va.) were cultured in Dulbecco's modification of Eagle's medium (CELLGRO, Hemdon, Va.) supplemented with 10% Fetal Bovine Serum (A TCC) and 1% penicillin/streptomycin (CAMBREX, East Rutherford, N.J.). Cells were grown in an incubator at 37° C. and 5% CO₂. The cells were plated on two sets of uncovered and covered devices at −40% confluence, for 16 hours with one set and 20 hours with the other, to contrast progression of apoptosis over time as a result of DOX-ND elution from the native and porous devices. DOX (2.5 μg/mL) served as a positive control for apoptosis, and culture media as a negative control. Cell harvest comprised of a PBS wash and subsequent lysis in 500 μl lysis buffer (10 mM Iris-HCl, pH 8.0, 10 mM EDIA, 1% Triton X-100) for 15 minutes. 30-minute incubations with RNase A (313 μg/mL) and proteinase K (813 μg/mL) that occurred at 37° C. followed the buffer treatment, separately. The samples then underwent phenol chloroform extraction, followed by DNA isolation and precipitation in 2-propanol at −80° C. for at least 2 hours. After washing with 70% ethanol, the samples were resuspended in water and loaded onto a 0.8% agarose gel in sodium borate buffer, run, and stained with ethidium bromide (SHELTON SCIENTIFIC, Shelton, CI).

### Example 4

### Compositions and Methods for the Fabrication of Hybrid Parylene-ND Films

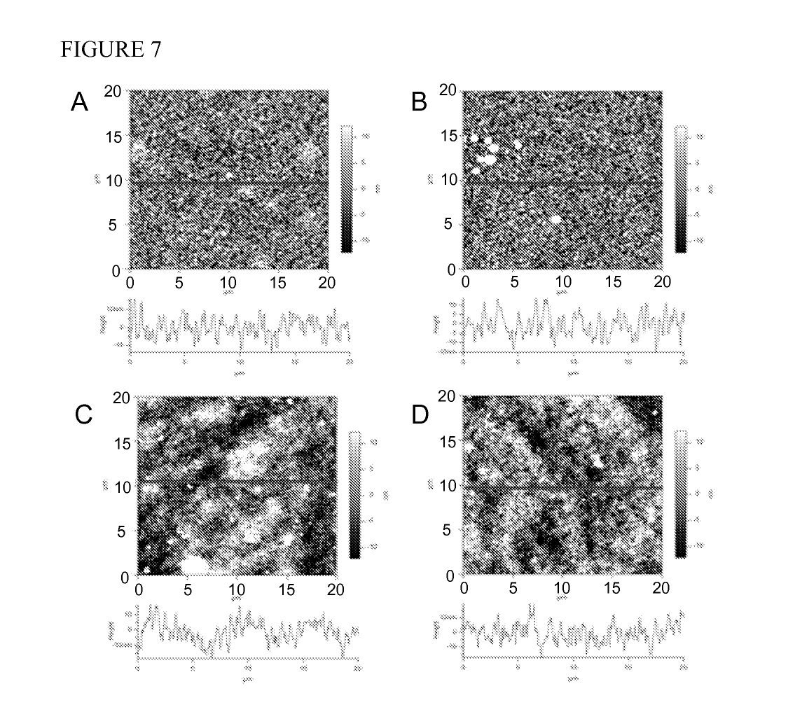

For elution and biological assay studies, a conformal and impenetrable base layer of Parylene C was deposited atop pre-cut glass slides. Within the Parylene deposition machine, the Parylene C dimmer (di-para-xylylene) is pyrolized into monomer form (para-xylylene) and then deposited at room temperature in a vacuum, conditions under which the monomers spontaneously formed polymers (Gorham. Journal of Polymer Science, 1966, 4, 3027., herein incorporated by reference in their entireties). The process was carried out under ambient conditions, hence, the functionality and structure of the DOX-ND conjugates were not harmed or inhibited. The base layer formed a flexible foundation upon which an implantable patch could be constructed, and simultaneously provided an impermeable and pinhole-free platform for unidirectional drug-elution. Newly deposited Parylene is hydrophobic (SEE FIG. 7A). Additional surface processing was performed to enhance drug deposition uniformity and elution. The Parylene layers were oxidized via oxygen plasma treatment, which has been shown to increase surface roughness while adding CO₃— and carbonyl (C═O) groups, effectively creating a hydrophilic surface (SEE FIG. 7B) (Lee. Journal of the Korean Physical Society, 2004, 44, 1177, herein incorporated by reference in its entirety). Oxidization of Parylene C surfaces have been shown to be stably hydrophilic after treatment, while increasing the level of cell adhesion (Chang et al. Langmuir, 2007, 23, 11718., herein incorporated by reference in its entirety). Appropriate amounts of a DOX-ND solution composed of a 4:1 ratio of NDs and DOX of concentrations 330 μg/mL and 66 μg/mL, respectively was then added to the base layers via solvent evaporation at room temperature to produce a final concentration of 6.6 ug/mL in solution (SEE FIG. 7C). A second ultra-thin Parylene C layer of patchy porosity was then deposited as an elution limiting element (SEE FIG. 7D). At diminutive Parylene dimer loads, film deposition could not be guaranteed to be conformal and pin-hole free (Spellman et al. Journal of Plastic Film and Sheeting, 1999, 15, 308, herein incorporated by reference in its entirety). With smaller dimer masses, the dimensions and amount of pinholes were increased, acting as an adjustable physical barrier for controlled drug release. Furthermore, this additional thin film provided a structural platform that simultaneously protected the underlying DOX-ND and acted as a base for additional device modifications. This base layer-drug-porous layer configmation has been applied towards studies involving dexamethasone and triblock copolymers in order to assuage inflammation. The final layer of Parylene C was also treated with oxygen plasma.

### Example 5

### Assessment of Slow-Release Characteristics of Hybrid Parylene-ND Films

Samples were immersed in nanopure water and placed in a 37° C. and 5% CO₂ incubator, to simulate physiological conditions. Upon incubation, the pH of nanopure water reduced to a value of pH 3-4. The decline in pH enabled a greater release of sequestered therapeutic than in standard room temperature, although the present invention is not limited to any particular mechanism of action and an understanding of the mechanism of action is not necessary to practice the present invention. Experiments were conducted during the development of embodiments of the invention to assess the slow release potential of the Parylene-ND based patches.

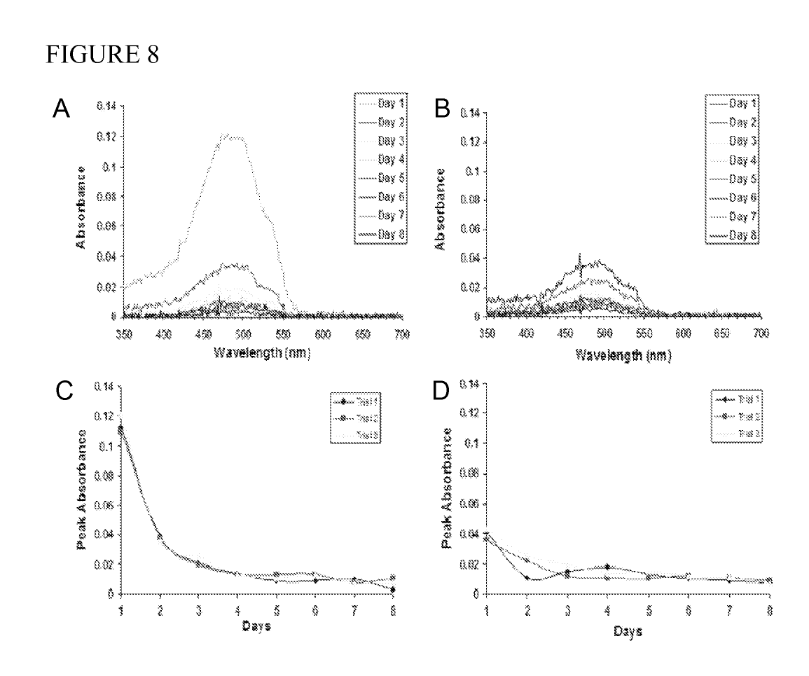

DOX solubilized in water generated an absorbance signal from approximately 375 to 575 nm, with a peak at approximately 480 nm (SEE FIG. 8A). Absorbance values under 350 nm were not recorded since Parylene C does not absorb strongly at lower wavelengths.

Films that lacked the deposited thin Parylene layer eluted the majority of the deposited drug in the first day, while films that contained the layer demonstrated a more controlled and constant release of therapeutic (SEE FIG. 8B-C). Controlled and localized elution offered several advantages over conventional systemic drug administration, including the ability of maintaining a desired concentration over long periods of time with a single administration (Langer. Science, 1990, 249, 1527., herein incorporated by reference in its entirety). Moreover, it is contemplated that DOX has poor penetration into tumor tissues, due to low diffusion rates caused by small interstitial spaces and strong intracellular binding (Lankelma et al. Clinical Cancer Research, 1999, 5, 1703., herein incorporated by reference in its entirety). Due to this effect, steep DOX concentration gradients have been observed when injected in vivo, with the highest concentrations localized near microvessels. It is contemplated that a gentler concentration gradient can instead be created with continuous treatment. Drugs with poor penetration, like DOX, also have been shown to have low cell death thresholds, as they only affect cells located at the periphery. Maintained continuous treatment could alleviate this obstacle by eliminating additional layers of cells through lengthened time periods, without supplemental therapies (Tannock et al. Clinical Cancer Research, 2002, 8, 878., herein incorporated by reference in its entirety), although the present invention is not limited to any particular mechanism of action and an understanding of the mechanism of action is not necessary to practice the present invention. As an additional benefit, sustained release can prevent oncogenic regrowth between chemotherapy sessions.

Small dosages of drugs applied through continuous infusions have been demonstrated to provide a gradual increase in penetration while reducing additional toxicity effects. Large doses of DOX administration have led to nausea, vomiting, alopecia, myelosuppression and eventual congestive heart failure. With a continuous infusion of DOX, patients experienced lower cardiotoxicity and reduced occurrence and severity of side effects than normal, even at higher cumulative dosages with no noticeable effects upon drug efficacy (Legha et al. Annals of Internal Medicine, 1982, 96, 133., Legha et al. Cancer, 1982, 49, 1762., herein incorporated by reference in their entireties). It has been contemplated that cardiotoxicity might be related to peak plasma levels of drug instead of cumulative drug dosage.

In pursuit of the benefits attributed to slowed continuous release, the hybrid films were tested for their initial release profile over the first eight days (SEE FIG. 8B). Uncovered DOX-ND complexes eluted at least three times more DOX over the first day than films with the additional elution control layer, which released drug at a nearly constant rate after 24 hours. It is contemplated that the muted initial release of the covered films aids in reducing symptoms associated with spiked levels of drugs that result from direct drug administration. After eight days, there remained a great deal of DOX-ND complexes on both uncovered and covered patches upon visual inspection. It is contemplated that the residual DOX-ND was due to large aggregations of NDs surrounding an inaccessible DOX core or physical entrapment onto the uneven coarse oxidized Parylene surfaces.

In order to evaluate the long term performance of the patch, the experiment was repeated over a month-long period (SEE FIG. 8C). A similar initial trend comparable to the eight-day results was observed. The initial surge in elution of DOX-ND from the uncovered film affected drug preservation and dosage. The decreased ability of the uncovered film in sequestering drug was a direct cause for the increased elution over the first three days. Samples were allowed to elute for a period longer than 24 hours at specific times in order to determine extended dosage levels, namely at days 12-16 and 22-29 (SEE FIG. 8C). During the aforementioned periods, covered films eluted a greater total amount of drug than uncovered films, primarily due to the uncovered film's drug reservoir being exhausted at an early stage from its large initial release. Since equal amounts of DOX-ND were coated on both films, the increased elution from the last data point demonstrates the covered hybrid films will elute for a longer period of time than uncovered films.

The decreased drug preservation resulted in increased initial drug dosage, which is important during the nascent phases following implantation. An immediate release of drug following implantation has been shown to cause several negative side effects previously discussed. The ND-Parylene nanofilm device is envisioned to circumvent these effects because the initial elution of DOX from the hybrid film is gradual and tapered, rather than abrupt and rapidly depleted. The robustness and stability of the Parylene based patches were confirmed visually throughout experimentation.

Based on the linear relationship equating spectroscopy measurements with DOX concentrations in water (SEE FIG. 8D), the approximate dosage of the eluate was determined. Uncovered DOX-ND devices initially eluted nearly 90% of the DOX-ND complexes into 2 mL of nanopure water over the first 24 hours to a concentration of approximately 575 μg/mL, with a steep decline in elution to 400 ng/mL after a week. Conversely, samples with a thin Parylene coat maintained a more constant elution rate, ranging from 2 μg/mL to 450 ng/mL in the same time frame. It is contemplated that this continuous elution can alleviate drug loss through blood circulation, extravasation or other methods of excretion (Tannock, Cancer and Metastasis Reviews, 2001, 20, 123., herein incorporated by reference in its entirety).

### Example 6

### Assessment of Biological Performance of Hybrid Parylene-ND Films

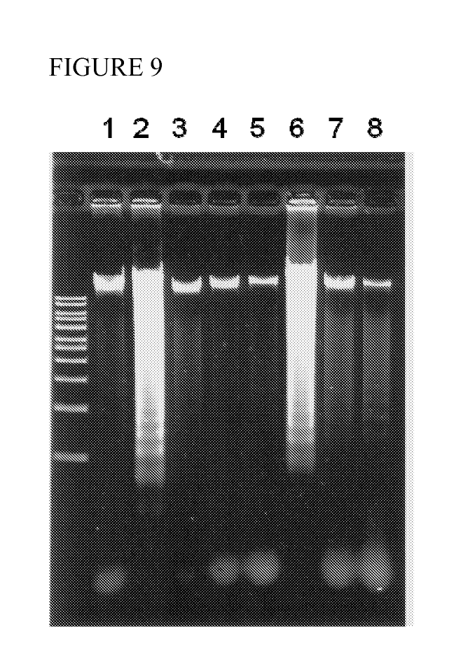

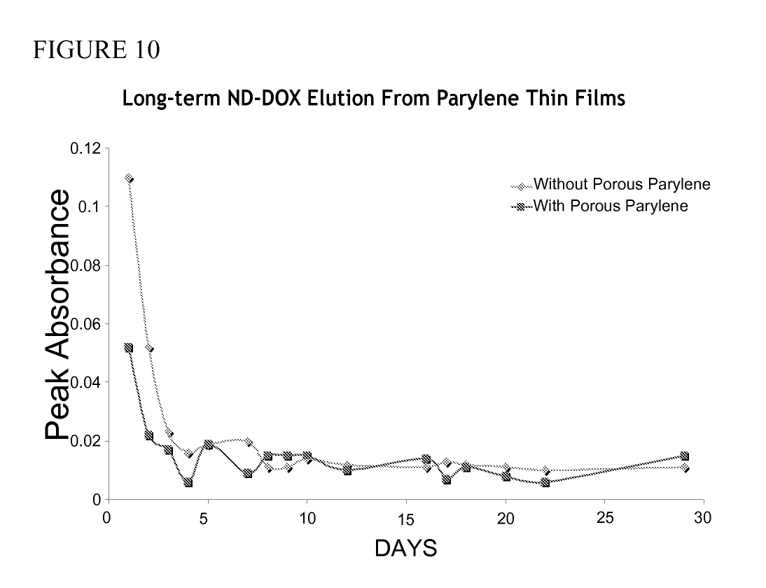

Experiments were conducted during the development of embodiments of the invention to examine the biological efficacy and mechanisms of the nanofilm. DNA fragmentation assays monitoring the presence of apoptotic responses were conducted with RAW 264.7 murine macrophages in various culture conditions. RAW 264.7 murine macrophages were selected due to their natural recruitment to foreign bodies such as implants. The patterned degradation of DNA that is characteristic of apoptosis appeared as a result of exposure to DOX, and was observed in the gel for the positive control and all samples containing DOX-ND (SEE FIG. 9). Two sets of samples were harvested after 16 (lanes 1-4) and 20 hours (lanes 5-8) of growth. Lanes 1:5, 2:6, 3:7, and 4:8 correlated to the negative and positive controls, the uncovered device, and the covered porous device, respectively. The ability of DOX-ND complexes to naturally reduce DOX elution rates was seen when comparing lanes 3, 4, 7 and 8 to the 2.5 μg/mL positive controls. Whereas positive controls prompted rigorous DNA fragmentation, the DOX-ND devices displayed a more gradual and delayed onset of apoptosis, which can reduce the severe side effects that result from a sudden spike in DOX dosage. In addition, the DOX-ND devices were loaded with over 13 times the concentration of DOX compared to the positive control. Therefore, the assay additionally attests to the slow-elution effects that are native to the DOX-ND complex. Furthermore, the relative degrees of banding in the gel suggested the onset of apoptosis is dependent on DOX dosage. This interaction was most apparent when comparing uncovered and porous devices at lanes 3 and 4 (16 hours) with lanes 7 and 8 (20 hours). At 16 hours some fragmentation had occurred in the uncovered device as a result of greater DOX-ND elution and a higher concentration of DOX-ND in solution due to the lack of a porous Parylene layer, which further inhibited the onset of apoptosis. At 20 hours, some fragmentation had occurred in both uncovered and porous samples. The fragmentation assay correlated with the spectroscopy data, revealing the different relative rates of elution from porous and uncovered substrates, further demonstrating the sequestration abilities of the film, Moreover, the data demonstrated the ability of the device to deliver at least 13 times more drug to a localized spot. The combined effects of localized delivery and gradual therapeutic elution of a large reservoir of drug offered a safer, yet more enduring and potent drug delivery device.

### Example 7

### Compositions and Methods for Preparation of Hydrogel Carriers ND-Conjugated Therapeutics

DOX-ND conjugates solutions were fabricated in accordance to protocols (H. Huang et al. Nano Letters, vol. 7, pp. 3305-3314, 2007., herein incorporated by reference in its entirety). Upon overnight ND ultrasonication (100 W, VWR 150D sonicator) and filtration (Millex-GN, 0.2 μm Nylon, Millipore, Billerica, Mass.) to remove large ND aggregates, ND and DOX solutions were mixed thoroughly in a 5:1 ratio of 12.5 mg/mL and 2.5 mg/mL concentrations for NDs and DOX, respectively in an aqueous 2.3 μM NaOH solution.

A 200 μL precursor PEGDA (Sigma) solution consisting of PEGDA, the photoinitiator 2,2-dimethoxy-2-phenylacetophenone (DMPA) dissolved in methanol, and nanopure water was mixed with 100 μL of nanopure water, 2.5 mg/mL DOX or the aforementioned DOX-ND conjugate solution for negative control, DOX:PEGDA and DOX-ND:PEGDA hydrogels, respectively. The resulting solution consisted of 50% PEGDA, 1% DMPA and drug or drug-ND conjugates solubilized in water. These solutions were then mixed thoroughly and photopolymerized for 2 minutes with a handheld UV lamp (UVP UVGL-58, Cambridge, UK) at 365 nm exposure. The resulting hydrogels were 5 mm and 16 mm in thickness and diameter, respectively.

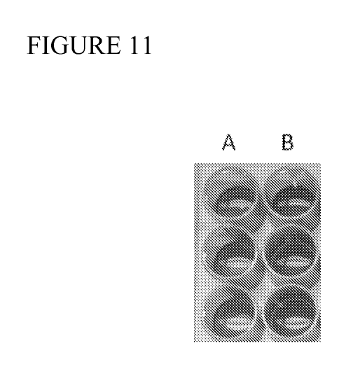

Upon fabrication, hydrogels were incubated in 1 mL of either nanopure water or PBS in 24 well plates (BD Falcon) and repeated in triplicate within physiological conditions (37° C. and 5% CO2) (SEE FIG. 11). At 24 hour intervals, the eluate was collected, refilled and analyzed via UV-vis spectroscopy. Release characterization was straightforward as solubilized DOX generates an absorbance signal which peaks at 480 nm.

Characterization was performed with a FEI Quanta 600F Sfeg Environmental Scanning Electron Microscope (ESEM) in low vacuum mode (1.20 Torr) at an accelerated voltage of 20 kV. ESEM is well suited for characterization of hydrogels since samples do not have to be coated with an electrically conductive material. In addition, since ESEMs operate relatively higher chamber pressures, hydrogels can remain hydrated during imaging (Zheng. Advanced Functional Materials, vol. 11, pp. 37-40, 2001., herein incorporated by reference in its entirety).

### Example 8

### Hydrogel Carriers and ND-Conjugated Therapeutics, Methods of Use

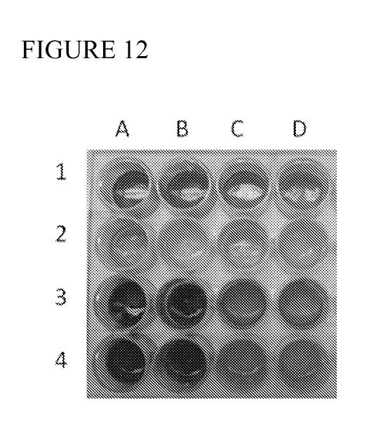

Experiments conducted during development of embodiments of the present invention demonstrating the sequestering abilities of ND-PEGDA hydrogels were visually analyzed (SEE FIG. 12). Hydrogels that lacked NDs (SEE FIG. 12, Rows 1, 2) rapidly released the majority of the drug after 24 hours. In comparison, hydrogels encapsulating NDs (SEE FIG. 12, Rows 3, 4) exhibited no significant visual evidence of drug release even after 10 days of incubation. The results were consistent across varying PEG compositions.

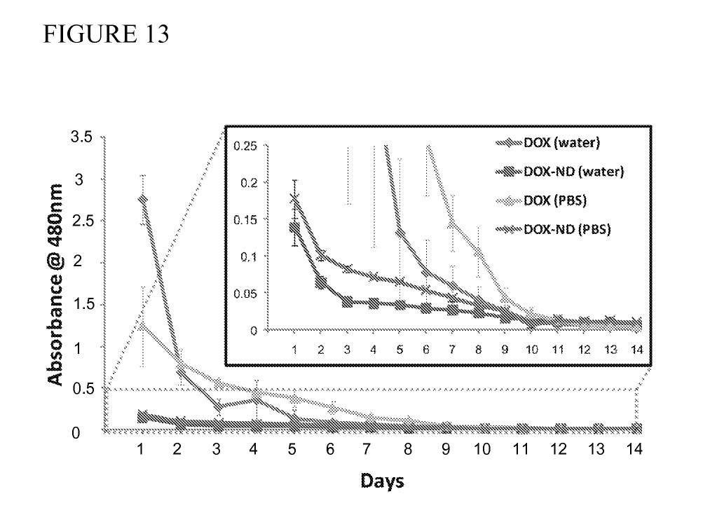

Elution results demonstrated that DOX-ND:PEGDA matrices released diminutive quantities of drug as compared to DOX:PEGDA samples (SEE FIG. 13). Within standard DOX:PEGDA hydrogels, the majority of the reservoir of drug eluted after a few days while ND:PEGDA hydrogels retained the majority of DOX for at least two weeks. PEGDA hydrogels lacking NDs incubated in PBS released the majority of the drug within the first week as compared to the first few days as with hydrogels incubated in nanopure water. DOX is less soluble in PBS (Pierstorff et al. Nanotechnology, p. 445104, 2008, herein incorporated by reference in its entirety). This effect is evident within the few days of incubation, as ND lacking hydrogels initially release about 20× more drug than ND:PEGDA samples in nanopure water.

There are several advantages to avoiding the ‘burst release’ common to many hydrogels (Huang & Brazel. Journal of Controlled Release, vol. 73, pp. 121-136, 2001., herein incorporated by reference in its entirety). These include, but are not limited to: reducing drug concentrations to under toxic levels, maintaining and extending lifetimes of drug release devices, prevention of any wasted therapeutic and avoiding unpredictable initial therapeutic release profiles. Although, methods have been developed to limit burst release, they require additional complex steps or major PEG compositional changes (Catellani et al. in Migliaresi, C., Et Al., 1988, pp. 169-174., Lee. Polymer, vol. 25, pp. 973-978, 1984., Wheatley et al. Journal of Applied Polymer Science, vol. 43, pp. 2123-2135, December 1991., Lu et al. Aiche Journal, vol. 44, pp. 1689-1696, July 1998., herein incorporated by reference in their entireties). In some embodiments, the present invention provides a reservoir of drug that can avoid burst release without the addition of complex processing steps towards hydrogel fabrication.

Hydrogels containing NDs exhibited more uniform and constant release across two weeks. The majority of drug eluted from the ND lacking hydrogels in a seemingly diffusion-based manner (Gayet & Fortier. Journal of Controlled Release, vol. 38, pp. 177-184, 1996., Sawhney et al. Macromolecules, vol. 26, pp. 581-587, February 1993., herein incorporated by reference in its entirety). Comparatively, hydrogels containing NDs released small amounts of drug at a near constant rate after the first 24 hours. Within standard hydrogels, release profiles are initially governed through diffusion. As diffusion effects wear off, hydrogel degradation mechanisms reliant on hydrogel composition dominates, eventually becoming the central factor responsible for the majority of therapeutic release (West & Hubbell. Reactive Polymers, vol. 25, pp. 139-147, 1995., herein incorporated by reference in its entirety). Since ND:PEGDA hydrogels sequester drug extremely well and avoid diffusion-based release, the small amounts of drug are hypothesized to be mainly due PEG degradation. It has been noted that if mesh sizes are decreased, then slow-released can be enhanced, but at the expense of increased brittleness (Scott & Peppas. Biomaterials, vol. 20, pp. 1371-1380, 1999., herein incorporated by reference in its entirety). By dispersing NDs within the matrix, the flexibility of the hydrogel can stay intact while simultaneously providing slow-release.

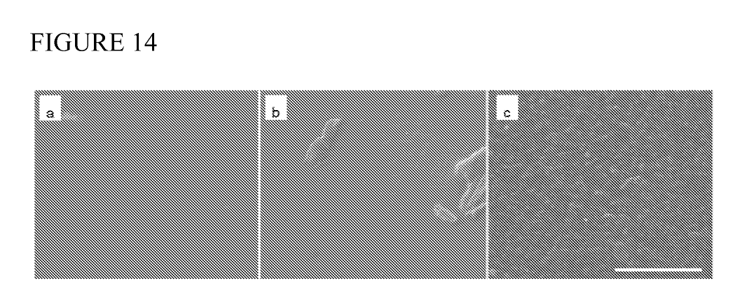

A uniform dispersion of NDs was observed due to the in situ incorporation within the PEGDA precursor solution. ESEM topographical images compare PEGDA, DOX:PEGDA and DOX-ND:PEGDA (SEE FIG. 14). The dispersion of NDs within the hydrogel matrix is easily observed (SEE FIG. 14C). This method of mixing the agent within the precursor solution prior to photopolymerization has been utilized in hydrogels to generate uniform dispersions of bioactive materials within the polymer matrix (West & Hubbell. Reactive Polymers, vol. 25, pp. 139-147, 1995., herein incorporated by reference in its entirety). Photopolymerization has several advantageous attributes, namely suitable and simple processing ambient conditions, limited byproduct formation, and elimination of a need for toxic catalysts (Zheng. Advanced Functional Materials, vol. 11, pp. 37-40, 2001., herein incorporated by reference in its entirety). Photopolymerized hydrogels have been accredited towards being a promising material for tissue implantation, replacement and engineering applications (Anseth et al. Journal of Controlled Release, vol. 78, pp. 199-209, 2002., Xin et al. Engineering in Medicine and Biology Society, 2006. EMBS '06. 28th Annual International Conference of the IEEE, 2006, pp. 2091-2093., Bryant & Anseth. Journal of Biomedical Materials Research Part A, vol. 64A, pp. 70-79, January 2003., herein incorporated by reference in their entireties). As hydrogels can be formed via photopolymerization in situ, hydrogels can adapt, be molded into complex shapes and adhere strongly through attachment of microtextures within the tissue complex. Experiments conducted during development of the present invention indicate that ND encapsulated hydrogels provide a localized source of slow drug release for biomedical applications (e.g. adjuvant therapy helping to localize and slow release therapeutic on the surface of tumors or damaged blood vessels and tissue). In some embodiments, smaller doses provided by the present invention may aid in minimizing side and toxicity effects of particular therapeutics.

We claim:

1. A nanofilm composition comprising:
(a) a base layer, wherein said base layer is composed of Parylene A;
(b) an elution layer, wherein, said elution layer is composed of Parylene A; and
(c) a therapeutic layer, wherein said therapeutic layer is composed of at least one therapeutic agent, and wherein said therapeutic layer is between said base layer and said elution layer.

2. The composition of claim 1, wherein said at least one therapeutic agent is selected from the group consisting of: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents.

3. The composition of claim 2, wherein said anti-inflammatory compound is dexamethasone (DEX), glucocorticoid, or an LXR agonist.

4. The composition of claim 2, wherein said anticancer chemotherapeutic agent is doxorubicin (DOX).

5. The composition of claim 1, wherein said elution layer is semi-permeable.

6. The composition of claim 5, wherein said therapeutic layer is configured to elute through said elution layer.

7. A nanofilm composition comprising:
(a) a nanodiamond layer, wherein said nanodiamond layer is comprised of nanodiamonds functionalized with at least one therapeutic agent,
(b) a base layer, and
(c) an elution layer, wherein, said nanodiamond layer is between said base layer and said elution layer.

8. The composition of claim 7, wherein said at least one therapeutic agent is selected from the group consisting of: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents.

9. The composition of claim 8, wherein said anti-inflammatory compound is dexamethasone, glucocorticoid, or an LXR agonist.

10. The composition of claim 8, wherein said anticancer chemotherapeutic agent is doxorubicin (DOX).

11. The composition of claim 7, wherein said base layer comprises a Parylene compound.

12. The composition of claim 7, wherein said elution layer comprises a Parylene compound.

13. The composition of claim 7, wherein said nanodiamond layer is configured to elute through said elution layer.

14. A composition comprising:
(a) a nanodiamond element, wherein said nanodiamond element is comprised of nanodiamonds functionalized with at least one therapeutic agent; and
(b) a carrier element, wherein said nanodiamond element is contained within said carrier element.

15. The composition of claim 14, wherein said at least one therapeutic agent is selected from the group consisting of: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, inhibitors, free radical scavengers, chelators, antioxidants, anti polymerases, antiviral agents, photodynamic therapy agents, and gene therapy agents.

16. The composition of claim 15, wherein said anti-inflammatory compound is dexamethasone, glucocorticoid, or an LXR agonist.

17. The composition of claim 15, wherein said anticancer chemotherapeutic agent is doxorubicin (DOX).

18. The composition of claim 15, wherein said carrier element comprises a PEG hydrogel.

19. The composition of claim 15, wherein said carrier element is semi-permeable.

20. The composition of claim 15, wherein said therapeutic element is configured to elute through said carrier element.
